SEQUENCE LISTING (11 sequences)

SEQ ID NO: 1 - DNA, 55046 bp, Homo sapiens
agtcttctctctcgctctctccgctgctgtagccggaccctttgccttcgccactgctcagcgtctgcacatccctacaatggctaaaacagcaatgggtaaggcactgcgcctcgttctccgtcggctctacctggagcccacctctcacctcctctcttgagctctagaagcattcagagatattttataaagaaaaagatgttaatggtaacacaggaccaggaaggacagggcagttctgggggaggtgggagggcagagaagaggtctatggaaatctaaagcgaagaatttcttttaaaaggtagaagcgggtaagttgccctcctatgggtagagaatttattctgtttccatatttaaaattaggactcaatcgtgaggggaggaagctaccttaactgtttgccttaaatgggcttaagggacattttggaaagtgctttataacgaccttttttttttttatttcttctctagtttaagaagaaaataggaaaggggtaaagggaaggtgggagaaaggaaaaagaaaattgcaaagtcaaagcggtcccatcccgctgtttgaaagatgggtggagacggggggaggggatggagagaactgggcacattttacggtattgtctcgtcgaagaaaccgctagtcctggggtgcggtgcagggaggtaagacggcgggggacagggtgggggtaggacctccgctcctttgttttagggcaagggaggggaaggagagaggaagtcgcggagggcgtggagggcgcgggtgggcagctgcaggggcggggaagcgcgcggcagggaggggtggagggacagcggcttcgaaggcgctggggtggggtttctttgtgtgcggaccagcggtcccggggggaggcacctgcagcgctgggcgcacaatgcggacagccccacccagtgcggaaccgcgcagccccgcccccccgcccggtgctgcatcttcattcgaaagggggtcgggtggggagcgcagcgtgacacccaggagcccaaccctgcggggacagcggcgccacgccccgcgctccccgctcccgactccccgccgcggcttccaagagagacctgaccactgaccccgccctccccacgctggcctcattgttctgcttttaagagagatgggaaaagtgggttaacatttttcttttcggaagcaaattacatagagtgtttagacatagacacagataaagggttctttgaagacctttgatcgtttgcgggaaaagcttctagaacctagacatgtgtatgtataataatagagatgacatgaaatcgtatataaagcaaaagaggtcaaagtcttaagttaagccacgcgaaatttccgttttgtgggtcagacagtgccaaatatcggcaatttcataagctcagagagacaagacagtggagacacaggatgaccggaaaagattctggattcagggccttcatccgcaattggtcttgtgccttgagtgcccacggttctggcgctcagtggccccggggtgaaaaggcagggtggggcctggggtcctgtggcagctggaagcacgtgtcccccgggacttggttgcaggatgcggagacagggaaagctgccgaaaggactccatctgcgcggctccgccctgccctaccctccccgcggagccggggagacctcaggctccgagactggcggggaagaggaatatgggaggggcagttgagctgtatgcagtcctggaacctcttttttcagccccgcagtccacaacggcccgagcaccccttgatgtgcgcagacccccggcgtggctctcagccccagcaccgagcccctcccagccaagcgggtggctctgcagaaaagctggctcgagccccgcccggccacacaaaggcgcggccccacccagcccgggcgcgagaccgcagaggtgacccccttcccagggattcagggagggctgtctcttctcgcccacccacggtccgcggagctcggggctttttttcccccagcccaagccccccgcccaccctctgttctctatgattttccagaatggagaccccgcgaggggcttctctaagggagaccctcgctcctccagcggggcgcggctcggccccacccctcccagctgaggcccagagccgcctaccgctggccgggtgggggcgcacgtggcgactgggtgtgtggagcgcagccagccctgcagagccccgcgccgcgccctgcgctcccctccccggagttgggcgctcgcccccgcggtgcagccggggagaccggtttctgcgcagtgtcctgagctacccccgctttccacaattcgcagttcactcgcacgtccagaaaggttctgagaatgggtggtgggggcgatctcgcctcgctttctgcacccctcagaaaggtttccgctgcaggctagtggctgcaaactcatcgtcatcatcagtattattatcatttcaaatcgttgttattatttaatgattcagtagccttgtttgttctcatttgttcaaaagggacgtggattgctcttggttaaggattaacccttgttgcgttcgctttgcttcctcctaattgccctcatccctttcccccacaaaaaggtaaatttgtctccagttgttcattttaagttataaagcaaatatatttttgcttcctgccaggattatgtatgttcatgtggctaagatacatgtgcaagtgcttgctaagagcagggtttgtgtgccaacgattgctggaaaattctctgcaaagaattgtttgtggctgcaatgggtgagaatacacatatataattgagatgatcttcaacataaggttatatctataaatatataaatatagtttatgcacaaaattttaagttttttcccctgaaactgttcttccaactgctgattcttgatacagcctcaatcctacacagatacatggatcgtgaaatggtagccgccatccaaataaaaatcccaccccaaatatgacaaacgcaagcatcctttctggccataatttaactgcatttgcaaatcatgaaaaaaacactacttctgcagtattaaaataatagattttgaaattaattccaatttcaaagataattaattatcagggcgagtgcttttttcctgattcattaaacaattatgtattcagcatgattgtaagaggtgcatataatattccccattatcttttctaatgaagtgggcaccttctgaatggatatataagtaactagaaatgaaaagctgaggatttggtcagaatttcaggataaaactgaaagaaatggcagtagtttatcaattaatctcatgtatttagtttataccaggtgagtaagctgagcctgcaataaacactctctgtcccagtgtaacacgtcgcaggtagctagaatgataggataaattaatagaccttgtggtgtttgtctatgcacgttaaaattctctgagagaaagtatattttaaaatgataattaagattggacatttgtgctattaaaatctacaactttagtcaaaattcacaatggtttttttttacaataatgtgacttacagatttgtagtaaattattctattctaaaagagaaatgagtgtttttattgttacagctattacctcattaatatttttagcaaacttttatttgttgcattgaaagcagttttaattactttgggtttttatttttcaaattactaatggatagatggtggaataagcatttaatcatttggcacaatatgacttccatcaaatagctcattctcagtgattaaaaaatgctacaagaggctacaatttactcagattcaggaaatgtcctttcagagtgccataaggctgattcatataataaaatagttttcttccctataatttaagatcaaatagttacttagttctgtgaatacctagcagtagctatcaaacagaattttaaagttaaatctgtacaactaacaatgaagtggaggatgaatcgatacatattgaatggaagactttgtcattgataaattcaggccatctttaggaaaattccggatttatcaatcaccattattttttacttcaactgagtgtgactgatcacatgctcaggctaccttggtagctcattgctcacaggaggctgaaaaaagctggcctccgagcaggaggaagctcagagcacaaacctaggcctgggcgtggccactgggagctgctgatagcgaaccccagctcacaccagtttcttttttggtcgtgggaagaaaaacacatattatcctgttgtcacaagatctgtgaccttatatgaaaaaatgctagaattttttcattaaaaaagaaaatactgaactagccagtgacccagatgttttcagaacctagactggttctgtccattggaaaacctcggtgtctgcattaacttttcaccacactagagggcaatcatgttctctaaaaaagcagatgattgatgtaaacctagttccaaatattaactgtttaataaaatcttttcttttaccaggaacattcaagtgtttattcaataagctgatgccatgctttaccctagtggatgaacagagcttgtacaattttcaaggagacaggatgaaatgagtggtcataatctgaaagtagatacacgccctggttaattattccctgatggttttacttctcagttttattacattgttattataataccatttatgttacttctgagattttgtagtggataaatagtagaaaaatgtcagtagtaatagcaaagttatttagcagccgaatattttaatgcttaaaaataaaggaataaattaaagaaaatcattgtttacttcttcatcgattgaaatgtgccccctgttcagagcacatctgaatatcagagtctccacctgcagagaacatgcagcttagcgagtaaaacaggcaggtatgtgatactgaggaggtgtaccaaaaactgactgctgttatttttcccatcttctaagtctgtctttcttttccatttaaagatacctttttaaatctaatccaatgtgatttcaatctagttttatcagatttcaacaattattgagcatctccttgtagtggttttctgtttattagaaaatcgatgttaattttaacgaagtaagaagaaatatataagtataaactaattttgggtatcatcaaaagtggattttttaaatatgcattgatagaattattttttgattacattttatgtaattctaatccagctataaaatatttaatagtgtcatattactgtgttcctcaaactttgatgtgcatatgaattacctttgattttcattaaaatgcaaattctgattcaatacatctggcttgaggcagacattctgtcttccgaacaagctcccagatgatgctgattctgaccactaaacacatcagttttagggatattaacttgtaatatacaggtatccctcctggtaagctctggtattatgtcttaacatttttaaatctatggtaatctttacaaaatattttacttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcctgaataccatgtgagaaaattcttaagaatttattgagtatgactgtatatttgaaaagagtgttttcttctgcttatctaagccaataaaggatcttcattattcaattctaactttctaaggaagtcaacctacagatcagaaagaggatcttcaaggaatagcatcaaagacatagtcaggtctcccatgcagtgactggctgaccatgcagccattaccacctttctggaaatattatgctgcaaaaatgatacaatacacgaaatatctcaaattaaaaaatataacatttcccaaatagggcactaaaaacatgatcccaaataaaactagcttcagggtttgcagaatatactgttactcaacacaaagttggactaagtctcaaagttagccattcagttgttgttaacagttcatttcagggtctctcagaagctgggaaactttccatttttgcaatttcttgtacattgaaggaaaggaagacacacttaagacagcattacaaaagtaattcatgttttaaatgtttaattctggcagtcgggcagggctctctgtataacctcatttggagatgacaaaaatctaaacttgagggcctcgagccaataagtcttcctatttctttactcaaacattttcccgcaatggtgctttctttcaactgtttttctggtgtattcataaattccagattctctatgggaagtaacttttattgattgatttaacccttgtatagcacatataacatgcaaggcattgttctaagaactttccacatattaactgtgttaatcacttaataatcctaagtaggttctattacagatatggaaactgaggcacagaaagttgaagtatcttactcaaggtcacacagttagtcagatccagaatttgggcccaggccatctggcttcggaatccatctttcaccgattgctgctagtctcatatctgttccatgttagaggtgagctcccattgcagaggtcacacctgtgatatcaccattttatttaaacagaccagagatggtcttctcctttctgatcacagactcaccttgaagagaaaatacttccaaattgatgcctagttttaatagcttacctggggcttattcaaataattgccatgatttaggctttgggagaaagagagctatgaggccgtgtgggttgtaacgtatgagacacatggcgttctgcaggctcagcacagcatcgatttctggtgggaacacactctgatgaccagttccagaaataacattgacttaatctcctcagtcccatcatggttagcacatttcaaaatgcctccttaactacttccataggccagagatatttagttttaacattttgttgaataaaataaatttacacattcacatttaatataactattagatgttatttcaagattctcttcatattaccatcaaagcaggcaggcaggcaggagagaactgtaggaaggttttgaatcccttgtgaaacatttttaattatcttttaataaaggaatcaggccctgtcatttgtcaaggagacatttgcagtagtaaagcttgtgtttataatatccatttttattagtcatgattaaagataacatttgtgtacatttgttctcacaaaacacttttatatgagtgtaaaggttaattaatgcatttcagccatcattttgctggtcatgtggaaatatagcttctttaggaattgtacttagagtaggagccacatattatactataaaaccataacaaaaatattttaagtttgttctcacttgttgttgacctccagagtaaaatatttaatactctggaaagttatgggtttcaaaatttattttatggcaagaaatagataattacagttctcatagagcacatttaaaataatttatttttatagggcaaaaatattgcctaggactgaatgatttttttttttttacaaagattgtaaagcaacgcctgcaagagtgcccatttagcagttattcttctggaataattgtattttggatgttggagttcgcacattaaccattagtacaagtacccaatataacaatagatcatcaggataataaatctgtccatcttttagttgtatgtctttatatcaggataaagagaattgagtgaaatttatctaaacctagtcccacaaatacttttacaagagagcatgttaaagtgtaaattaaatttttattagcattctactctgtctttggaagttttttttccttatgaaatgcagccataaagtttaacttccattaacaaagctgctcacagtaaacctattataataatagtttcccagtttgggcttcctagtgaggagcaacctaactcacacgaaacaaccccaacttataatatattgactgttacaaaactgagaccagaaaatcccatcaagatggtactgttatcatttccagactctcgggaagaacattaatcatctcaggcacttttaggatagacttattgcagcctccctgggaactctgcttcagaacataattatttttattaatgcagagttactttttatttccaacaaaaatatctattgttattatttaagtcttacagctttatctgagaaattccaattagcacccttctcataataaatattcaaacacatgaaaaattaccaaagttgttctagtcttttaatgacatattacatgatcctgcactcttgtcactttaaaaattatctttttattatatttctgatgatttttttcttatatagttttttaaaaggagcaggcaagcatagaagactaaaaaatgttcaaaagaaaaattaaatcgcatgatctatctatatgggaccttgtcatttttagaaaacattcacctgcttcatccttttgaatcttcatataatccctctgagatgggcatactatacaagttgtcttatttaaagattggtaaatttaagctcaaataatttattcagtggcaagcctcagaggcagactcggaacacaggtctaatatatattatatatatattataacatataatatatatattacatataataaagttgtgtatattatttacctatcaaaatatttatatgtaatatataaatatgttatatatcatgtatgtgcctatttcatacatatatacacattcatgcaaaataaggtttagcactccctccactgtcctgtaataaaacatgcacagtgagaatagtcatacacgaggcatatttgtcttcagtttaaagtcattgatagtcagtgtcactaactaaagtaaaatagattggagcaccaactttgttctgaagcctgtgccaggtattatgagaacaaaaataaaaatgttcctcacccttggtggatttagtcttttgcagaaaaaaagatcctgtacatgtcagaaagttcaatagtaataatggtaatttataactataaatggaagtcaccatctcacaatttcaccatcttaacaattttgttaaactgccctacaatattacaagatagtacataatgatacactagtaacatcaactaggaagtaccaagatccaccaaaaggctgaaaaatttaaatatttaatgagtccatcaaccaatctggccagagaattctttaattaaaatgcttcccaaattttactgagaatcagcagcgtttgaggagctagcctccacccccagaggttctcactctattaggtctgaagcaggtcccatggatttgcatttctaacaagctcccaggtggtgctgatgaggctgattcagaaccacacttggagtagacctaaaacagcagtgacctgtagggtccccaagcagcaggccaggacagcatgtgagttacgtcctctgtggagctctgcaacaaggcgtcaagaggtcagagtctaagtccccatcagctctgcccttctccaccagtgctgctggtgctgcatggaaggaagagcccagaagggattctgagtttcagtctttactcttgctgacgcaccttggtcaggtcaattttcctgtttgttcctctaattcagcatctgtaaaatagccatgtgaactgccttgtccatatcagagggtctttttcagactcaaggaaaaaaacgtgaaagtgattagtgtctgtcaagtagtatataaatgcaagaagttgagtttttaaattgtcattagatataaatacccatgtgcatgcatttagaatgagtaaagagggaacaaggagcgcaatcaaaaactgcgtcatttgctttttgaaaaatactttctatgtaatgaaaagtgaaataaaatgttaattgagtccctctgacaacagcatcagacgttttgcagttcttgtgattagaacccacctggccagcccttcttcctcctaaagaagagccttcttcttcttaaatgaaggttggctcagaagaagcaattaactcattcaacgttttgttacagtcaatccacatccaacttttccccaactcaatctgctttaagggaaggatggtaagtggtggcccaagatggcaaccatcaagcttagagaatctctagaagcaggggtgtccccagcaagtagacactgaaaatatgagagggctgataagccagagataaaactcagtacttactttgcttctagtccatgtctacccctttcttggcaccaccttgacactaccctctgagtccaccttcctgagatggtacaaactctgcttagacaaagcagcccatgtccaaaggtgttagggctcagtttaaagctgccttcaaaagttaaaacagaagtgtaaagttctgtgcaattaaaaataatcagcttgtcttggaactcaaacgaatgtaaaatcctatgaaaattaaaaagcagtaccacaagttaccccaaaagtccttaggtcagtaactgttcctgttacaggtaagagagagcatggattagaggtgggcgtgggtatccagtggacatggttttgaaccatgctccactactactcactatctgagaattcttaaatttattaatcatttctatattataattttctcagttatgaaatgggaaaacaatacctaaatcacatggttgttaagtaagcaattgattgttaagcatttggtcatcaaaaatattaatccccttccctgattccctagataaatgatgaaaatactaaataaaaataataaaaatttaaagtgaacatctcaattcttatactttgttaatttctacatgtattacaaatctactagaaattacttggaattgaggaaatgattactgcttaataattctttgtggtagagggagagttggtatcatatttatgagacagcagccaatatagtatatctcaaaggaaaaaatccattctacataatgccagaatttaatagttaagcattttatctaggtcacagcacaataagcaagatggataattaaaataaaagtatatttctcttgcatatatttctcatttcatgtttccctatcatattttatatcttaccttacttcaaatacatatataccttcaataaaactgagccttcttgcttacccaggaagtttcatcattcagtagaaataaaagatgactttagaaatattaaaatacaaaaatctacactgaggtcttttgaatgcaggaaaaagaattatatcacacacacacgtacacgcacgcatgcatacacacacacagaacctctcgttctttcttaacatcttatcaatccatcagtttcactcccactccgtatcacctgactgtgcacaatatctcattgccacctcccagtcttctccctgcctggcaccctcctgctctcctgcttccactttaaacacccttccttcagctaggtcttttctttcagggatcctcccgttgctttcttatctggatcaatttagccttcctcttctccacccattagtggataagcacgacaaagacactagagtcaaataatacaaacagaatataccttagatgagtatggtgatgaaaaggatatggatacttagagtttagcactattctctcagccactcaggaaagcaacgcctttacaatcaatagtgtttcaggtaccaatcaataatctgttattgctatttttaaaatctataaggtatcagtaaaatgtaattactagagcaacaaagatatcttgtgaaatcaaattagtattcatccagcaactgagtacaaaggtttaagggaggataactaccaataccaaaacattttaagcattttgttttgcctcctaaatatcaaatcatgtaaatgtgtggtacataaattaggaattatatttatgacatagctgcagacatattaagagaaatatgtgcttatatttacaagtatagtacagttctttttcatattagatactgttgatgataatctgcatataaaaatgctcaatattttttcacatttataagccataaaatacagctaataaaatgtgtttctactttctcataaacatggaatagtgacaaacaaggagctttatatgaaagcaccattacaatttaaactctcacaaggtcataatatattgcactaagcaggagagttcagcttatttaaaaaaaaaaataaactctaatgaggttctggaatgcagagccaaagcataaagatggaaataaaagaattgcatgtcttctgaactgacttggttgatgatttttttaaaaaaggttttgtgtcttctgacttggttgatgattttttaaaaaaacgttttgtggtagaacaaataaggtaaatgaaattcagtatttaggatgaaaagtttttctaatttcaggaacaacattgaagaaatattgaactaagcagctttgaaagaatcagattccatttgttgaaatttttctgagaatgaatttttttaagacagtgtacacagttgcagtgtgtattggttatggattgtggcaagctatattacaacttacccaagaaataaggaggctgggcgtggtggctcacacctgtaatcccagcactttgggtggccgaggcgggcggatcacgaggtcaggagatcgagaccatcctggctaacacggtgaaaccccgtctctactaaaagtacaaaaaattagccgggtgtggtggcgggtgcctgtagtcccagctactcgggaggctgaggcaggagaatggcgtgaatccgggagggggagtttgcagtgagccgagattgtaccactgcactccagcctgggcgacagagcgagactccgtctcaaaaaaaaaaaaaaaaaaaaaaaaagaaagaaagaaagaaggaaaaaagtcacttgaaaagaatactggactttgtgtccagcttgcatagctgaaaagaataaaaacctgtccacttaaactcattgcaaaaagaagatgtcactcctacaaatagcaaagagtcatgaaattattctatccagaaaagtatacatttcatccctttggataaattttagaagtgaactatgaatacatacggtgaggatagccagctaagaagtcaagaaggatttctcaaatttgctgctcagaaagatcatactctccacaaaacaaataatagcaggctttccaagtcaaccttgaatccagctttcctttatctttccttcttgtgaactttcactagtttactatctaacaatgaatttgacgatagccacataccatcttatagcaatatttgttatcatatcccttgttatttatcattcacctgctctgcttgagccagctacaagtcacatgtcccacgcactttttcctgtttgattttttacagcactttgagacatgtctcattattcctacttgacaggaaagaagccatggaaagttgagtgacttgctcctgatcacaaatgctggccaaggaagagtcgagtttcaaatctaatgatctttccactgcactctagattcctcattttgaactatttttttattttttgcactatagacttttttccacattttgaactgttttttattttttgcactatagacttttctcttatacccaactatattgatgacttcttttaggctagaaacttgtttcacttactttccctttcttcagattgctgcaatattggccaacatgtattgggtacttactgagtcaagtactgtgattgtgccaagtatcttataggaggattatcatcctcatttttacaggtgagaaaggaaaggaggtaaagtcacacacagccaacaaaaatggtagcaccaggatttgaaacaaatcagtctgacccaagttgactttgttaaccactgtatgcacagtcttcttagacatagtaagagctctaattgtgtttggtgatttgattattatgacaaagtaagtaagggaagcagggagaattataagaaataaggctccacaacacttggctatagcaaagccccttaaaacttcaaaaggtcacccaaagaataaagatcaggctgggagcagtggctcacgcctgtaatcccagcactttgggaggccgaggtgggtggatcacctgagttcaggagttcgagaccagcctggacaacatggtgaaaccctgtctctactaaaaatacaaaaattagctggatgtggtggttgccgcctgtaatcccagctacttgggaggctgaggcagggagaatcgcttgaacccaggaggtggaggttgcagtgagccgagatcatgccactgcactccagcctgggcaacaagagcaaaaaactctgactcaaaaaaataaataaatcaatcaataaaataaagatcaatttggagaaattaatgcttattaataagcaatgtcttgcacagcacttcagtttctcaatacattacctaactcaatccttacaacaacaccctatccccattttgtggataaataaactcatgttcagaaggttgaataaattatctaaggttaatagttcctgacctagagctcaaatcttcagtttctatcatattcttgcccttaccctggggtagctaacattcactcactagtattggagctaaaataagggagagaacatataaatgaatacaaaggagacattcacctgccttctctttctccttacatagagaaggttgattatctgctattgtgaagtttgccttttgaaggatagaaatgagaagactttcttaaattttgcctctacgccaagaaattagagtggtaccaccagtagttccattttcaaactatcactgtagctaaagctatgtggtaagggccaaggaaaagaagtattcttgcacttcaaaatgcactgaaataccagtcagtagcataatataaaggaatttagtggagagaagagttgacctcaatctggctccaacatctcggctcttaacccctaccctacacttgttcttcatggggaagctaattgggccactggaagattcagcagctaccatttgcagctgagggacagcccctccctgcttagcaaccaatggatatgcatttatggaacacctgctaactgcgacacacactcctatgtatgagggaaaatacaaaaaatgttaaaggagatgccttcccttgccctcaggaaacttaagtatagttgcaaagaaatgattagcagcaaacgaaaccatggagaagtaagggctaaggtctgtgaaacaagcctagaaaataaccttgtccttgaaaaacacaaaaagaaagaaagaaagaaaagaaactccaaggcccttgtgaaggaaaccattaagtttgcttcacttctgtgtttaggaagacacaaacccagtcttaatgaacctcaaggccacaactactggagacatttaggaattgtcaccacattctaatgtatatatcctctgtttggcccttcctattaatattttgtaaaatttttgaagatatgagcaatgtttaaaaccatgaatccccctttttttataagtaatatttaggctgaataaacaagagaaaataggacataaaggggagccaacgtgtgccttcatttataatgtattcccaagttgtgagtttggtttatcagcaatttatcatgccaaattccaagtcatatttatctatgcagatcaaacacttgattctatttttgccttaatttttttattgggtatgtttatgaccaagtcatatggtattttctgtgacagataaaatgcacaggttattccaatctggctcagccagtcatagcaacatgtagtccttctcatgtcttaagaatgagtatcaagaattcaaagggagttccagatggcatccaaaaagcttacagtttatgcatcacttattctaacagtagaaaaagaatatttgaagccaaaaatagaccttgcatgtagcatgtggaagagtagaaattgccctgatagttaaacaatttgaaattcaagacattaatttctttatgaagcatttgtcacatcataggtaatattttatgcctatcatatatatacttattatgaaatacaaagaaattattcattctatctaagactttgtatcctttaccaatatctctccattctcccacctccaccctagcccctggaaaccacccttctactctctgcttctatgagttcttttttagtgagatcatgcagtatttgtctttctgttcctgtcttatttcacttgacataatgtccttcaggcttatccatgttgtcacaaatgacagaatttccttcttaaggctgaatagtattccattgtgtgtatgtagcacattttctttattaattcatttgttgatggatactcatattgattccatatcttgggtcttgtgaataatgatgcagtgaacataggagtgcagatatctttttgacatactgattccactttgatgggatatatacccagtagtgggactgctggatcatctagtagttttatttttttttattttttattttttttattttgagacagagccttgctatgtcgcccaggctggagtacagtggtgccatctaggctcactgcaatctctgcctcctgggttcaagcaattttcctgcctcagcctcctgagtagctgggattacaggcacgcaccaccatgcccggctaatttttgtatgtttagtagagacggggtttcaccatgtctcgaactcctgtcttcaagtgatccgtccacctcagactcccaaagtgctgcgattacaggtgtgagccaccacgcctggcctagtagttctgtttttaattttttgaggagcctccatactgctttccataatggctctaggaatttacattccaccagcagtgcacaaggattgcttttctccacattctggctaaccagtctcctgtctttttgagaacagacatttcaacacgtgtgagataatatctcattgtggttttgatttgcatttccctgatgattagtgatcttgtgccttttttcatataactgctggacattaatatgccttcctttgagaactgtgtatacaggagaaaataatcacttctcagaggagctttcatttcaaaatatccgggaaaaaaatagaaaaaatggaaaatttatcctagagtaagttgtcttttatattttgaccctgtttgtgacataaactggatgatacaaaactggaatgcaaaggctttaggaggattacttacttacttgtatattgctttaggttgtttgcagaaaattatactaattgaagttcaggctatgatgtgataaaatctatgtcaggagatgagtctacatgcaaagtttgaggaagtgacatttgagtttcaaaacaaaaaagcaattttcaatgtcatatctaggttaacccaaaagatttctttcaccctatttagctgcctctaagatggatgctgaggataattacactgtagaacaataggacgatgcttcacactcacctcacaggctctgttattcccacatactgccagagatactccaaaataaaatcactgcaacatcaggcagttataaacctcaacggtattattttctatttatatacagtatattttatattttacaagtataaaatagaatatatttattctattctctttgacacaaagtgaccataagacatattacttaagtatgactagcaaagtcatggggcttgtcattcaggaggaaactcttaactaactgttcagtttttgttcactgcaccatttacataagccaaactaatgcttcacactgtgcaaaacaatgcacagtgttgtgaatgaatggctaaaataaaactctaatgagtggggtttgaaaaatgcaactttagaaaactgttgagaaaatgttgcacactgcgcattttacaaaatttcgttgaaggacactggatattctttttaggattatggagggaagcaaaattttggctcctacatgcagtttttgtggcctttgcctgaaatagtcatctcccattaattatttagatatcattcatttcctaagacaacatttagggagactgccttaagtacaatttgtacactacccagataagaattctttttggtgaaacatcgataaatattacttggcagtaacaccaagttaaaatatttgtttcacagtcgacgttaataactattatagataaagtgaattttataagacatactcagatctaaaacagcaatatggagctcttcaaatccattgaaacttcataccagcctacggaagtagaggtttttatgcaaactcttcaagaaatatgctctgaacttttaattccttagattgatagaggaattaaatcatgatataactaataggtttgtggtacaaattgctgctgcttaatctgactctgtgtcttcccagtgttctatatgaattagatattccattatctaaagacaatcaaccccatcccacggtgatagctctaggactccctttgagttcattaaatctgtattctcagtctccaaacttctggttaattcaaacagaaaagtcaactggcccatgaactaaaataaagtcatctgaattttttttttattttgcagtgtgataaaagtctcgcactttttatttctgaaagtttctgctttcactgagagcataataggctatccacccttatgcaatcttacatacaaagtcatagtcaggctaaattcaaaaacacatgtgagatagaagtcaacgtttattttctggagaaaagccacacattacaacaaagtgaacaatgaagctggcatccttatcactggtgaccaaaacatttgtgactctggacattggccccacaaatgcgataaacattctgcataggaagtgagttttgctaattaaaaatggatccaaaatactttctactcttcagccaagaattaaaaagtaatagggaggaattgaaatcacttgggtgctacattgagccattctggagaagcaattcagagaatgtcatggcagcctcaaattgctgctcaggagcatcccagcttagaagattgcaggaaaggaagagcaaagtcattcttacatgagaactgtccttaaccagatgaatagactctccattttttaccctggctttgtctcatttaagtcccaaccaatctagctatcattttaggttttactacctgctagtatttaggagcttagggggataaaaaaatccctcaatactcagaattagacttggtgataaaaatcttgacacataaacagaataaagcgctttcattactcctctaaaccacagtgtcatttggtctctatcaaggactgtaagaatttctttcatcaggggaaagaaaaaaaggacaagagcctgcaagatgtagcggaactctcattaaacacagcaggagctttaactggaatccagagtaaggtgaggtaccaggttacaacaatttactgcttttattacaattttgatcacaaggactgattcatgtcatctagtttcttttccttgtcactatcactggtgctaagaatacatcaaattgaaatttaagagcctcatatgtttctgtataacccagtgatgggttgtactgctttgaccttcttaaatgtccctttatttcatttgatatccattcccatagaaaaactataatgctttggttggtcaaaatattaatctttcaaaacctccctggcttagaaaaccaaatttttgtagagagagatgggtagaatctaattttattctaaagcaattagcattacatcatcacagcagaaatatctagaatattacctcatgtcagtgatcttctgatatgttaaaaagggtattttaaaatctgagttatttctttttctttttaaagttacatcattaattacatactcatcaaccaaaatattttatgctccaaatttgaaccgatatagtatgtaagaagtgttcaaaatgaaattattttggtctattttgtctttgaagaagatcacagggatggacctcccaaaaggatttttaaatgggattacatatctgacttttaaaaaaaattatctgaccttgagttatagtgccccaaagtaagcaaagttccaaacacacagtatcatcagaattgagttaaaattatcaccaggggcttaatttctgaaattaaaaaggaaatgttatttccttatgaaaagaaaaggaaccaaaaatgaacttcaaggtagctgatttctgtctatgttaagacttaggtaatgggagaaagggaaaaggaaggacagaattaggagaggagcagtgtttaacaattgcgggtgcaagactcaagttttttagaatccattagcagagaaccctatttctcccattaactgctgtccttttaaatcctggcaccagctctgaggactgcagggtccatagctagtgccccactctacccagtttaaagacaccactgcctggaaatgacaggggtttttttcttaaggaaagaggtgctttctgccacgtatatataaattggtaagcttcaaataaagtgcttttgtcctttctgtctatcagaaactgtgcaaatcgaattgctgtaaaaccaagggcaagagacatcaatcctgcattctatagcatctgattttatcctttatccccaggcacatttcaaaaggaaaaaaatgaggttgcatttaaattgagtatttgggacttgccaggaaaacctcccgctagactaatatgattgcagggaaaacaagagaaaggaaaagtggagagggagtgtgctaacagatcctgggcctcgtcagcagagccgtcctgagcacaaggccatggtcagacatctggtcccgcgaatgacgttttctttatggtcattaagaacaccagtgtgtcgggacacaaacaagtattcctttcagggattatgacacattttctcccaaagtagtatattaatgacatttccagagcattctttactatcttttatatgtgatcaggaagactaatacatatcactacttcttttacacacagcattagccaaaactaaagtgtcaaatacaattttgcctaggatgaataaacagaagaaatttttatgatactgcactatcaattccaaattaaataacaacaaaatgataagtgttaaaattcatattaatgattgttcccacacaagccggaaaaaatctttctaagaagtctttcatgagttaatcccatctttcaaagtgttcagtggctccgaattcagttactgtttcctatcagttcttctttcattaagtctcttcccttttttttctctttgcactatttcccttagccgggtacataatctgctgtgctttattcatttgtgtcttaagtttgtttcccgatgacatacctttccagcaacgccatctggggagtttgggcaactgtaccacgttaggaggaaacccttcttcacaggagagtgtgcctttgctgcagggaaggaattaggatttgcttggactgtggttgcagctggcttttaaggatctccttagaatgcaagcaactcatcaatgagaatctctgcaatggttgtcactgggtagagtcatgctatgtggggtcatagcctttgaaacaaataacagtaaagataaaaatgctattaaaggaatcaccacccacagaggttaactgggttttgtccccagaccacctcgaacaagaaagaacatttttatcagtcattttcttagttttagctgataaaacaaagtaccatagactaggtggcttataaacaacagaaatttatttttcacagctttggaaactggaagtctgagatcaggccgccagaatgatcagattctagttagggcctactttgcttttgcagactgccaacttctagctgcattttcatgtggcaaaaggagattgagctagctctctggtctcttcttataaggacactaatcccattcatgaaggcttcaccttcatcatctaattactctccaaagaccccacctccaaatactatcacattgggaattagatttcaaatacaaattttgcggggacacaaatattcagtccataatagtaatgattactcattatacatagggctctaaatgtgctagcttctgatagtttttacactcacttctctttattagcttgtcaagcataattagggcagtggccttactgaaaattattgaatttagtttcctaaggacagatattgaggagttttttcttcactaaaaattcacgttccgatacagctttcatctgttactactttgtgagatggaaaatcttttattttatttttatgtttggattgacccttcttaataaagtcggcatgtaatatgcttcatgtgtttctaatatgtgcttaattttgcaaaatgttttgcataccagaatgcatttctcttccaaaaaaggtaccagcctacaaaaccttgctgttactgttttcaattagttcatggaattaaatgtattaaatgttttatgctctggcagaaattatgattctcacttaactccatataaatctggatctgcctgggcctttataagtgacacaatttcattaactgaataaacaaatgatacaaagaaatttggtttagccttctaaaattccaaaggcgttcaacaaaatatctcagaatggatgttccaggacttttatggcacaggacaacatgtattgcttattttaagaaaataagctaaatagtgaggggattcttttagcagatcctcaggatgtgttaggttgaatcataggcaaatgatatttgatcattgcacctgttaacacattgaacctcatcctaaaattgtagagctagaagaaagccttctggcagtttttaaatagattgatttactgcaatttatccagaagcttcaccgttgtcactggctacatgtgactttggcctctgtggggctatatcctcatttgtaaaattggtggtgaggtaggtggacagttgactaaataatctcttagaataattctagtatctgtggatctaaagcatccaggggttgaatatgtttctttctggccaagaaaagatgcacctgtcaataatgcccaaactcatcttctgagaatcctctttcccaagatacccactctcccttgggttatattatagtaatgatcagaagcccctgccaagaagaaactgttaacctgggaggtctatattttatttcacagccatctgtttatactttctcacaagttagtgcacagtatacccatcattttctaccattttccttaatttattaattttactaattgcataattaacaaaagtaagaagattttacctccttatccccatctggtagtttgcagatacttggcctgatgacaactgacagtgatgagatactcaccaagtttaccagggcaggaggcttcctagagaaaaaatgagaaaatgaaatggggaaggggagtgaaggattgaggaggtgacaatctggactcttgcaactgcatggcaaggttggcacacaagctgggttgcaacggagggaaggagatccttatcagatgtaatcagagctcagatcgagggctttggtgtgtgtagaaagagggagagacaaagaacttaaaacagagctgccatttgaccttgcaatcccattacttggtgtatacccaaaggagaataaatcattctattaaaaagacacatgtgcttgtatgttcatggcagcactattcacaatagctaagacatggaatcaaactaggtgtccatctatggcagattggataaagaaaatggggtaaatataaagcatgcaatacaacatggccataagaaaaaatgaaatcatgtcctttgctgcaacatggatgcagttgggacccataatcctaagtgaattaacacaggaacagaaaaccaaatacagcatgttctcacttataagtgggagctaaacactgagcacacatggacataaatatgagaacaataaacactgtggactactagaggggggaaggagagaggtttgtaaaactacctatcaggtgctatgctcaatacctgggtgatgggatttacaccccaaacatcagcatcatttaatattcccatgtaaaaagactgcacatataccccttgtatctaaaataaaacttgaaattaaaaaaaaaagaaagaaagaaagaggctggaaatagaggctcacacctgtaatcccagcactttgggtggccaaggtgggtggattgcttgagcccgggaattcaagaccagcctgagaaacctggtgaaactctgtctgtacaaaaaatacaaaaattatccaggcatggtggagcgcacctgtagtcccagctaatggggaggctgaggggggaacatcacttgagcccaggaggtggaggttgcagtgagctgggatcacaccactgcactacagcctgggtaacagagcaactctgtctcaaagagagagaggaaagaaaaaagaaaagatggacagataagaaaatgcacttggagattaagagaaagcagcaacataggaccctggataatgtgtttgcttaataactatcctgatgagttatctgactattcccaaatgagtacgtggcaattcaggctgaaccatcagagtagccctccggaatcttacttatgtacaatagacctgcatgcacatttactagaatgagcctctctctctggtaatcatgtctgcttccactaattccatctgtttcctctctctccctcctatcctgctagatcttaattccttcgaccttcctttgtttttctaactccctttctttctcttgttatttaacctgctatactatgcaattgatctcctctgcactaaggaacatgcacttcagaattctgttgacatcttgcattcctttatatttagtgaaagaatgcaaaggagtctacctggcaatattcactctgcaggaggcaataattattattcaaattaaaggaagcagtaaagagaaattcagaaaaaatgaaatatactaatcttcagcttttcatttcagcctacaaggaaaaaatgaaggagctgtccatgctgtcactgatctgctcttgcttttacccggaacctcgcaacatcaacatctatacttacgatggtgagtaacctaggatagacatacccctgctagctagatcatttggaaaggttgacatatatttgtttcttacagctcctgatataattacatcaatattttgtagctctcactattgacttgccgtgtctagctattatgtccaattgattacctattgctgaaaacagtttgaatttggtgctaataacaacacatcaatgtctgttaagaaatgtggatggattcttattaacagccacatccagcatatcaacatccacaatatgtctaaggtctttctttgcaaataatttaataggctaagccataattggagtagatcataatttgtaagaaaatgctttatacttagaaaactcaagagaaagaatcaacaaccataattgtttttgctttattgtagtctttataaagtttctatactttgtatatacatgtcaaccagctaatgataataataattggctcaataaataaaactgacttacgactgaggccctagataaagagggtctgaaaagaaaagcctaaagaattagcatggcaattaacatgattgaggtgcaactctttaggtttgatttatcctgattcattttgcttactttggctctgccacaatccacatgatcttggtcaaatagatacttggattctctaagtctcatttaactctagcatcttcctcttggagttgttgtgaggtttaaacggtttaatgtaagtcaaatatgcaaaaccaagcctagctcattatatcactctacaatgatagctatcattatcaacatcatccttacctaattcagtcaatttaactaaaatattttatacagttctatgtatcctagatatccctaaggcatattttactaactctcaggctcacaaatatttttcttttccatatatgtaaagaaagacattaatgacaaaacaaactgaccttgtggcagttaaccccttctgcacctttaaagcctattcaaggactcaaaggcatttaccttccaaagttattctatcgtagcacaaaaatcataaatgctaattaactgttccataaggaaatgtcctccatgtgaaaggaattctgtctccaaacaaaacattcattagaatgcagggccaatgcctactttgtacaaattcattcggtcagcaaataaattagacagacctttattatttgctagatgtagctgtgaagaaggatccagctatgtttcttatgagactaatgtcgaactatgggttgtcactgaggatccagagttccatagggcgtagtcctcaccttcaaagaattcagggcttagtagaagagtcttacacaaatgactagaatgtagaacacagagtggttaggacaaaggagccagggatggtttttgctgggttagggaatgaaaaaaggggaagaaaatatgtgaagttatgtgtgagctgattcttgaaataagctgtttttatttgcctgcgttctcttataatccttttccataggcttccataatttttattgagctgtatttaaagttgaatagataattcaacatttctcgtaaactgtgcttcctaaaagagtccgtagagaatttcaaatttctgcagtctttaacttgacctggtatttctatgttagataataacgtgacttgtttattgcaggcaaacattataacaataaattattattattgtttacatttgtaagcactaagtatatggcttgtgctttgcattcagcatcctttatcatttaatcttcacaaccaccttagaaggaaggtactctttttatttccatcttttaaatgaggaaataaaagcataaagaagttaattaacttacctagtgtcacacagctattaagaggggcttactatttggatgcaaatataggcagttctaattccagagcctctaatctaaggcatttaaaaccccatcaccttatcaaataagctgtttttatttgcccgtgttctcttataatccttatccataggtttccataatttttataaaattgtatttaaaatttaagtataatcttggatgccatcaggaaaatgaaaaacatttttacatttgtgaaggaaaaagcccacatcatttccaatatagttattgagttagtattatctagactatctattagcagctaaggatctgaggtcaaggcctgccagcctggcattttacttgaccacaacctccatgtgcactaaccaggctgctaaaagaacattaacgggaacataacctgctggcttggttgccacaattttaaaaagacgttaataaattagagagcacttagaggttaggaaataatatggtggtaaagatctagaaacagtgtcattctggggcacttgaagatgtttagcctgggggaacaacttgaaatggaacataactgttttcaaatacttgaaaaatggtggtgcaccacagagaatggcctaatcatgggtagcttcagacttcaaacaaggatcagtgggctaaaaccagagagatggagtttgggactcaaagaatgctcatctgaaattgagggctgaccagcgaggttcttttaaaaatcattgcattttactaaattgtgagttctgtaattataaatgtcctagcaggtgctagctgtcatcttttctattataaattatactattttatgttataatttgtattatacaggcttaaaacataagggtctgataatctgcttatctttaatacataagccactgatagaaaataagtggctaaccattcttcagttctttttttaattgacaaaaattgtatatgtttgcggtgtatggcatattttgaaatatgtatacattagagaatggctaagtgaagcaaattcacatatgcattacctcacacacctgtcatttatttgtgatgagaacaaaaaatctactctttcagtgattttcaagaatacagtacattgttattaacaatagtcagcatggtgtacaataagtcttctgcggccgggcgtggtggctcacgcctataatcccagcactttgggaggccaaggctggcagatcacgaggtcaggagttcgagaccagcctgaccaacatgctgaaaccttgcctctactaaaaatagaaaaattagctgagtgtggtggtaagcgcctgtagtcccagctactcaggaggctgaggcaggagaattgcttgaacctgggaggcggaggttgcagtgagtcgagatagtgccactgcactccagcctggcaaaagagggaaactccgtctcaataataagtctcttgcatttgttcttcctgtttaactgaaattatgtattctttgatcaacatctccccagtctccacccctaacccctggtaaccacaattctactctgcttccgtgagttcaactttatgaatagtccacatgtaagtgagatcatgtggtatttgtctttctgtgcctagcttatttcacttagcatagtgtcctccaggttcacccatgttgtcaaaaatgacaggatttcccccaacttttttaaggctgaacagtattccatgtgtatgtgtataaattagattagtagatgttgccactccctcctccaccacagtggctctatccctggctcctggctccagccgagtacactagaggaggatattctaaacagcaacaacacaggagcaaagacattacaatggggtgttgtcttattgcccccattagactgtaagcatcttgaagacaaggacccccatcacagagtgatgttgtcatccctggagtgggcactgtgcatgattgatgactggaagcaatgaacatacagaagggcaaaacagaaatcagcaggatgctttgcatttcagcattgactttgccaaatatgcccaactgttcagggagttacattggttctaacgaagctcctgtgattcctaagcacaggaatggtgataatatatataatggtgcatgcatatatacgcatatctagataatgatatctcattatatgtgagaactgaagaactccgttatgtttctcgtctaaccaaaaagggcctacagctacgataatttccaaacaaataaatctgtgctacttgattttcatgcaaagctcatatttgttcaaaaggaaaataaagcttaatttaaaatcaatttaggctatttttatctaagtatgcttaccgttattcaactccctgcagatattgtcaaatttctcaatatggtaaatatttattctgttaaaatatatccatagttacactaaagacagagaggtcttatatgttctaaacaacatagagcaaatgctcataaacagcattttattcctatctcccggaataacaacgctacttccaattgctggaatctaaattattaaaataaacccatgctgcaagctttgtatgcttaacattctcaaatgttcacttttcagatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaaggtaactttttccataggttttccttctctctctccctcccctgctcctccctctcacacactcgggcacacatgcacgcacacacacacacacacacacacacacacacacacacacacacatacagagagcaatgacagctgaacctgtgccatgccaacatgtataggttttcagtagacacagagccaggctagttggggtaaaaactgtaagatagatgctaattttaggctagccaaaccagagctctcagaaatccaaagagcttcagtgctctagtgccccttcccgtatattgaatccccttattataaaagcctcccttccctagaccatcaggcagaagcactgtagagaaaacacagccctggcgaactccagtggtggggaggggaagaagtgctgcttcctccctctcaggatctgtgtcaccccctttgtcaggcgtggttttccttggaattacaaattaccagatcttccctccaagatctttcctgcccagggtaagggccaagagcttgcccctttcctcttcagagtcccactgcctgccctggaagttggtccttccaagatcaggaccttctctgagttctttgaatatgttctttatctttttctaagacttgatggggatttttctctttttgccattggtccctgcttatattaaagagctttccttttgccaaatctttacttttccataatcacatggctaagaagagccaagggtattatttgagaacacttagaaatcctagggactgtgtacacaaacagaagttgtttgaatgtgtctgttccaaccatgtggttatggtagttaatcccatcaaggtactcacgatcatccaaaaatggaattcttttatgtaattcatccccacattgtatttcccaatattttttatgatataattttagaatcaggtaatcactaagaacatgttccctgcacagttttatgatgttttctctaaaaagtcagccaaaactttggacacttctatgttggataattaaaaacagaatgaagataatcctcctcctaaagattgaattctccaagagagaatgcaggacaaacacagatgtgctgtgtatagtatatgtgcatatatacatgcatatatgtacacaaatatgtgtattatcaaataatgaggctcaaacattagaaatccttagattaaattttctaaacaagaaaacactaatctttgtagttgaaaaaaaatcctcctatgatatgtaatatgctgatctcaattttcacctaagagtgatgttctccaaatgtccgatgagcatgtcatatatatatatatgaatttttatatatataattacaatggtaattggtatatagagatatctatattatagatatatatagctatctctatatattacatataccaattatagatataaatataacaatggtaactggtgtatatgtgatgtgtatatatgtatatgtataccataattatatattaatattgtatatatgccataattatatattaatattggtatatatacaccatgattatatattaatattggtgtgtgtatgtgtgtgtgtatatatatatatatatataaaatactagttatcattgttctagatttaaaaaacaggaacctgagctactaactcgactatatatatatatatatatacaggaagttgctttaaaacatttttatcagcttttttattgttatttttagctttattctcatagtaaagctaaaataaattattcaacattatcaaaactttgctgccagcagatgtaagcaatacctaaaacagtggagagcatgttgcacccaaagcagtttaagctctgacccaagcactggcatcttataggcactgggtagagataagagtcataggtcgacatatattgagatgctatgacttgattagaatatggagtcagtgactgaggtgaaattaaaactcaaaccacaattcaacatcctgatttaggatgttgctggtgtttctaggtactacacttaatttgaaagaaattattgaggataaaaaaagaactgggatcaacaaaattaactaggtgttcttataagagtccctgaggttactaattaatgaaactgataaagctcctgcaccctgacagcaagaaattatcaatgattatacatttaaacaattgaattgaactagaaactggccacatggttaaaagacatttacaaatgtaatcatccagtgttatgatgcccagaaaaaaaaaattccttagaatgctttaaaagccgtattccatcacctttccagttatttgttaaacattttgtaatgcaaaaataaccatatagattatgccctagtggtcgggttttatttttagttttttatggtttttttttgttaatggtagagttttaattaaaagaaaatacaactaattagcagaaagtgccaactttaaaaaatcactaattgattttattctattgggttatactgacttaattagcactaatttaaagaactattaattatctttaaagagtctttagcaagtgcatatatctcagtaattatgttagtaaggacatgcctataaccaaaacccaactcaactagttaaaacaaaaagcaaatatgtgactaaaaagtctaggagtggctacagcatcaggaacagctggatccagggatcacagtattatcagaaaactttctttcagtgcctgtcatctcttcctgcatttaactggtttcattatcaagaaagtttaatttcaatagtcagttccaaattatttttctcacaacttagcaactccagcagaaacagagcttctttttcccaatagtttaacaaaagtcccgaaattgagtctcaatggcctggcctggatcacaggcccaacccagaaccaatcattatggccaagaggatgtagtagtttgatatgctagcctgaatcacatgcccaccactgacctgcaaaggattttaggtaagatccctggggtaagaattgtggaggggtagttccccagaagaaaatcgaggtgttctcacaagaggaaggggtaatggatcttaaataaacaaaactatagatgtccacattttctatctataaatgtttagtgttactataacaattagaataattatttagttcatacactattcaatttgtatctcccttctgttgccctgttgccgttattttcttacagatagaatgaaaaatattaatctaggcagctctgtgaaacagtactgtccaaggaatataacgtgagccaggccgggtgtggtggctcatggctataatcccagcactttgggacgccgaggcaggtggatcacctgagatcaggagttcaagaccagcctggccaacatggcaaaaccccatctctactaaaaatacaaaaattcgcagggcatagtggcgagtgcctgtaatcccagctactggggaggctgaggcagaagaattgcttgaacccaggaggtggaggttgcagtgaaccaagatggtaccattgcactccagcctggatgacagagcaagactccatctcaaaaaaaaaaaagaaagaaatgtaatgggagccatatgtgtatttttaaatgttctagaagccacattttttaaaataaaagaaatatgaaatgaattttagtaaaatattcttcacccaatatattcaaaacattatttcaatatgcatgtaatcaatatagaagtattaatgagctgtttcacattattttattcatactaagtgtttgaaatccagtgtgtattttacgtttacaactcatttcaattcatgttagacatattcctagtgcctagtagccaaaggcagccagtagcacagatacggatattaaaacagaaaacacctagtgaataatggggaaattttaggcctaagtttttaaaatccataccagataattattcagattcaaatttactttgttttttcatatatattctttaaaaattacattaatatgggaactcagaaagttcaaaagaaatttccattctatggttttagtctttacattgtcagaactaatgcaagtgtgaagtttaggatgtactgtaagtaataggatcttctaaatctcatgccttcttcagctacctactctgtttctatttcagttcctcactgtggggaggggacttctctgaacctaggtttcatctctcactctcgttcatggtaaacaggttttcctttgtggcacctagcacaattagtaagtaattagtatttactggcatattagtatatatatgcatatgtatttatttaaccctatgtcttctactagattataaactccatgaagatagaacttgtcttttgtttaatagtgcttggcaatagttattactgtaaacattttttttctttcttattcaactcctgttagtcattgcctgagtactacaaatgtttttaagtaaattaataaataataactttcagggccaaatgtgaaagcggcaatatatagcttgttttgattttttattccaccctcccatcctaaaacaattatagtcactaagtttccaaatgacatctgaaattgcactaaggaaatcctagtctgggcaaaatcactcagtcaacagatatttatcaagcacttactatttggcaggccctgttctagacacaggggatactcatcaaacttacattccagtgggggagaaagagctaataaatacatacacagcatattagatgatgcaaaattagcaggacaaagagaactgggggtgtgggggtgaaagaagctaatattatatgttattattactatatataataatataattattggatagtcaaaaaaaaacctcttgaataagacatttgaaaagaagcacaaaggtagcaagggagtagggcgggcagctcttctctgggacctgaacattcaaaatgatgagagcagcaggtgcggaggccctgaaataggaatgtatgaggtgtgtttgagaaataacatggaggccagcgtggctgaagctgagagcagggggagagtggtagcaactgaagtcagaggtcacaattaaggactttgacttcacatgaaatgggagatcatgaaggataataaagccatttcactactttatgtgaatcacagcatctttttaaagaagtatccttttttaaagggggagatgactagaaaaataaatagtgttagataaatagagaaaacaggaaaacattctagactaagacagtgattccagaactaaggatccacagaggcgagaatgcagaaagtgtaggtttcagagcagtgggtagactaagggtttggactagtggatttggatagggagttggagagtagcgaggtgggattagggagggctgtgaatgccaggttagtgtgcaaactccattatataagcagtaaggagtcactacagacttttcaaaaatacatacatgttccacctggcccacgggttagcaacattttcgttgccctggacccatttccttcccaataagttacaggtttgtgaagattctacctagcaaacatattacttttaaataactattaataaattatcttaccatgattataatcaaaggaatctgtaattgctaattatttctgattattaaaagataagcagtattgcactaaattgacataattctaactcaaagtaaatatacagatagacatggctatagatgtgaaatatgatttctgttagggctttttaaatttaaaaaaacttacgagttctcctccctccccctacccttaataccttgaaggcctctttgtgggacttcagggaccccttcagggaactatgacctaggctgtatttggggggctttctgggtttatagctggaaggctgccacagaggcatcgccacttgggctcagattcactttgtgttcaatgttttggcaatgtccccacctccccattccatctgttgacactattgcagcactgaccatctggttactaggttggaggatactccctcgggctcctttgaaccagaattagtgctccagtgattagataatagaagaagcttgtcataaaaagaataagccctttccctgctttttctccattctttgattatcgctggtagtcagtgatgatcatctctatgagtctatatcaatctcatcaggtcagtttgaacctcatctcttgaaatcaaagtttccataatgcaactgacccacaagggtgaaatgacatgaatgctttaaccatccatttatcatttattcattcattcaaccaacatgtatttagcaagaggcagcagagttagcataactatacatcccagttggcccaggacaactccagctaactctcgttgttttgataccattattaattatttctctttactctcataagtgttccactttggacaatcaattacatgagcatccttagcagggcacagtgtttaagggcatctttaaaatattgtctttaagaacatgtggttaagagaatgtctgtgttcaaatcctggttccaccacttaaaagctgtgtgacctcaagcaagtgacttaatctccgtatgtcctcctttgtcaatctgtaaaatgagactagtaatagaacttatggagttagtgtgagaattggaaggttactctacaataaagacatataaccagcatggtaaaagggttagcaattactatgtgaagaagcatccagtttctgacctcacagagattatctagcaaactcatgattttataaagaaaagaagtttctcatcaacagagactgaaatgctaccatacaatatacgttgctttttttttttttttttttttgagacggagtctcgctctgccactcaggctcaggctggagtgcagtgttgccaccttggctaattgcaacctccacctcccaggttcaagcaattctcctgcctcagtctcccaagtagctgggattataggcacccaccaccacacccagctaatttttatatttttagtagagacaaggttttgtcatgttggccagactggtctcaaactcctgacctcaggtgatccacccacctcagccttccgaagtgctggcattacaggcatgagccaccatgcccggccaatatttttaaatattataaaatattctttatcaaattgcatagaagaaaagacagtttgataggtaatagatatataaataggtcaggccaactaaaagtgtcctgaaaaaattaatattgtgaaaacaaaaggattttaatgacattgataaaatctcaccctaaaagagattaaattaaaaatcaccctacttgaaccagttcagtgagatttcattagcatgctctcattactggcataatcagcttcaaagtcactaagcctctgaaaggaagatgtgttgcttattcttaataaaatggcataaaagtagatcattagtcaccaaacatgatagacttaccttttccatttgttggcatctcacattgtagatggcaattaaaatggaatccagggaaagagggggtggtttgtatagcaatggattatgaaacaaagtactggattattcaccgcttgacattcaggaaacattctgctccttacagaatatggcacgtgggccacagaatcttccgtgtgctaccttctcggtgaagaagagcacccccaagtttcttttcctaggagctaaccacagtaaacccattacacactttagcagaagggctcattctaaaggtcttaggattttaatcattttaaatttcctgttatgcttcaggctcttcaacacaaagtgaatattgtactctttggttttacataattatattcaattgtcatatttcaacaggacattatttgtgactttagatgggtcaataatgattttcattgtcagcagtaaagtcaataattacagacacatcacctaccctacttgtgtaaaagcattttttggtactaggagatttagtgtctgatcaacggtcctggatagcaagtaatatatcccccaaataatgaaaagtgacaagaaaataaatatgtttacttcagaaataaatggaaaattagtgctatctaaaatgtagtcttaagtctcatctgtgtacataaagtaaaatgagttttatgtactagttactcaaatttatcttccactccatttgtatagtaattaaactcttacactcagtaatatacaaattggtaattaacctctttgcaaaatgttaaagtgttcctaaatgtacaataagtctcctttcctgtctcattgtttttcgcttcacgtacctctcatgtaattatttcaatgattgagttcagtgtgaggaggtttatgcctagaaaaggtgctcaccaataacgtgcctcagttcccataatagcaagatcgagaaggttctttagtctcccggaacgtcacgttgaacatctcagttctatattttgccttgacatttgcattatatcagctgatcattgtcttgccctaattttcccttttaatattttagtgaccttctatgttaggtacaggttatttagaagtgttcctccaaggccagatactttttccttgaacaatttatttttaacaacttttagcgattttctcacttcaccaccctccgtttcataagtccacgcaatcacaattcctttctgctaatctgcacagtcaagatataaagtaagaatacctatttgaacatgtagtgagaactttacttctctgccaaaaatgaaggaaaatgctgccacttttgtatgtcacatgttttttattctacagcctcactcacttcatgtcatgttttagtgcagttttctggactaactgcttattttctcattgattaaactgcctatttgctcattggaattagagccaatttttttccttgagggtctgactagaagattaaactatgttcatgtgagaatcaatttctacctaagaaatgagttagaggagttatgggcagcaatatctatctggatgctacactgtgaaaaaggaagcgaggttatgcctttctaccccaatggggtagcagagacctcaggaactgaggtagatgcccccctggttattagcgcccctgaataatttgttcaaaaattgactgctggacaggtgtcgtgttgcacgcctgtagtcccagctgtgcaggaggctgaggcaagaggatctcttgagcccaggaatttgaggctatagtaaactaaggtcacaccactatactccagcctgagcaacaaagcaagaccctgtctctaaatttaaaaaaaaatattgaatgcttatgaatagagactaatataggaagtcataagtatttccttgggatagaatgctttccaccataattgacttgacatcctgtatttttgtatgtgtggacttaagttttaaatatttgaaacacagacaattattaagtcctgcaaatgtgtgagttaatagtggatataacattcccttccagggtgtaagaaaaggtaccacagaagtgagcagccctgaagcacagcctggcctagtttggcaggtctctgtgagttagcagcagactcacgtgaccacactctgtactgccttctgtttctgtttcaccccattaattgtgctaaagaaatgcacttgacacctatgctgtgtaatctcatttagccccaatagcaacaaaagtactaaccccattaaattgagtcatttcaaactgagccaaatgttgcactccagtaaatggagtaggcattggttataatgggaattctccattattcataatggaaaccacaggagtttgttcatgcagatcaaatgtgtcccaccaaggcaagaagtatggaaaagtggtgttgctgtattaccttgtaatttcaaagccttcccgtctgaatcttatttccctgctgtttcctcttgactttggttctttcacaaaggaaaattaagaacacaaatataaacattaagttaaaacacaactgaacaaagtgccaaacttaattggagcatctgaaaatgaaacattaggcagttgcagtggcctcttgataataattcacagtaactctctgtaagctgatcctgtctgaagagcagcaggcacaaggcccctggccatgaagtccatctcaaagggccaggctcagcaaagcaggatgcaaacccaggctttccaaataccaggttggggctcatgtcactgtgccacaggagcttctgtagaaaggctacttgaaaaaagtggccattaaaaatccaggtggatcctatctagggcagtgttggaaacactgatctatgggaggaggagcaggaaggaattgtttaaccactgagcagaaatgttacattgctacctgcctttagcagctgtggctgatgggtaccagttgctaagaagagcattacctaacagtgtattaagatagaaaaatgattttaaagcacggcacttagagaatgttgaagttttactttgctttattttgatttgtttggtttgactttgtctcctggagcatcctccatggatttctgttcattacaagagaaacctagggctctaacccaattcctaattcttggacacattgcacccttgttttgtgataatccagccttcttccttgagaaggtttgctggactggaggttacatgtattgaattttctaaaatgaaggtgcaaagctgtctcctcttatttctttgtggtgctcacttcactgtgagatttcctatcaatacagcccaagtcagtgggcatgcatgaggtggagatgagggagttaggaaggacttggactctcatcaaccatcaggatccctgaatccactaactgttcataatcaaagaagtttgaacaaatacttcacacacatgaaattgccaaaattttgcatttgagttgttataccagtaagtccagttgccatcatctccttgtcacaagtgtcttaaattttgcttttgataataatgattaccactcattcagtactaacttacttgatattagacactgcattaaataccttgcaaacattattttgtttgatcctgacaaccatatgagataggtactattcttatccattaccaaaaaaattaatttcatgaagacttttcccagagagagaaactttaaatatttacacacacacctctctccctgtaacaattccgtagtcctgataacagcaaataagcaaagtctgtgtaggatgctttaccaacagtcccacctagaggcaggagagtgaaccagctagaaaatattttattcatatttcttccagaaaggctccattggagtttgaactcaatttatgttataattttcttattatttttgtattggttttcctgaaaccaatacaaagtaagaaagcattggttccactaaaaatgtcctaaaaccagccaagcacagtggctcacacctataatcccagtactttgggaggccgaggcgggtggatcacttaagccaggagttcaagactagcctggccaacatgacgaaaccccatctctactaaaaatacaaaaattagcagggtgtggtagcacacacctgtaatctcagctactcaggaagctgagacatgagaatcgcttgaacctcagaggcagagattacagtgagcagagatcacgccactgtacttctgcctgggtgacagagcgagactctatctaaaaaaaaataaacacataaatagtaaaatgtcctgaaaccattatggggttaaagcaagaggcagggctggttcccaggattttctgtctaatctccagtgagccacagacctattcctgatcaacttgagaataaacacatcagtaaagatgtgtaaggctgtctgactttcccatttctgtagaattttatttgaagagaagtttctcctttctccaggccccatattgtttatacaaaaagacctttccagtaaatgtccacaaccactaccatcaactaaaatgttttcccactaatgctttcaatggtaatcagtatttaacagggcacttaggattattttttgatcaaccattgtttagatattcccacttataattactcctgtgaaggattgcctcggggcatcagctgatcctgagaaattatccagaagccatgagtgtgtaataatttagtcttaaacctaaataggtcagtattgggtgggacttttctcagctgcataatggggagaataaaaagaatatggaaagaagttacgtaacacatcctgggtcacaaacagaggtaagacttgaacacaggcctgacatcaaagcccatgccagtatgacttacaaaaggtagactggactacctgcatttgagtcactagtgatgcttatcactgggcctcaccaaagaaccttggaatcagaatctttggaggtagatgccaggcacctgcattgttatcaagtgctccagtgattaccattcactgtacagagccaaacagactcctgatgctggaagaaaattacagtgctcaaagtgcagggcagggtgtacatctggatctaaatcactgagcaaccacagggtttcaagagagggtcaaaacaaggactttctgctctctgtggccaaggggacactaagtttgcactgttctcagatctccaaagagactttggtgtatgggggatagggaggggggaagggggtgtgaaataaaaggagaaagtgaatttgattatttgattgatgaaaattgaaaagcttattgtagggcctagcctacagttgatgaaaaaacaatggatcaggaagaagatcagaacttgtctcagtcctcaactgttttcctcaggctttggttgaatattgccatcctgtaattcattatagcattttctgttgcataaacgcttagcaacaaagcctttttttaaaaaaatttgtaactcctcaatgaggattaaatgcttcttcttctaagacagtccgaaatatactcacagctgaaaattcagctaaccgcatttcccaactagccacattctatagaaaactctaagccatgcagatgagtacagacttgacaatagtgctcaaggctgggagtactattcatctgaaaagaatgctccctccaattggtgggccgttattctgctaggtttgtgtttggataattataagatggctatgtttttcttccccagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggtaatctcagcagagtcctgagcagatggatatattcatatgcagcacagctgggtgaacttccatatgcctgagcacagagacgaagtcaaaatttgctgcaggtgtgaggacaactaactcccatgggcagggtctcacagtgtagcattgagttagcaggaggtgcaacatggtagagaaatgggaatccatcatgaaagctggaattttgtcaaattttcccatggtgagtggattcagggaggctgattcatgcttttgaaatgtgtaagacttctatacaagcctcacgaggcaatctgtaggaaaaatgttacactggaaatattaatgtctatatattatattgatataagtataaataacatttgatttaatatttgtttaatatatgacattaaatatatatttaattaaaatattaaattagaaaaatatatttgccagaaaaggccagggtatttatgaacactggtaagcccattctagggtataatagcatcacatgggaccatagcaaagattagctcataggggatgtttcatccagttctggtatcctggtgcccttctcttcaacaacctaaacatatattcattcccatgagtcaggaggagctgtgctggagttcttctgaaaaatgctgtctttcacttttgtactctctatgctgtctcccacctatcccctcaaaaaacctttcctttgaaaatatacagtatagctgtgagtagtttagctgtgtccgtttccagaaattggaataagcattgagaaatgggatgtttgagaaagacgcctcaatccttttctgagcagtcagtcacccttcccgccagtagcaagtgcctttgtgtgataggcattggagatgcagagcaaaacaggagtgtgcctgtcatcagagccctgagagtttaattagatgagcctcctgttttctatttctcagagtttcatgtcttctgttagagatggcccttctcatctaaggttcaaaaaaccttatcctgaagttctgatgattctgttttcattctcagtctctgactgcaaatatccaactagaaacaaaggaaatcaggcatgaaaacttttaaagatataattgcatggagatcttcatttgtgctcgtgaggaatttttgaaagcattgctggggaagggtgtgtgggctctgatgcagcagtaagacactgaggctctcagaggtccgtggacgagtactgctgacttgggcaagaaccggaatagttacctgatgccttatccgaaacatgaaagttcggattaaatttgtatttataagctagtgtttttatactctcagaacaatgtcattgcgtttcacccaagtgagtcaagtcacgatttggaagaggcaacagaatttggctctctccaggtgatttatggcggtataggaacacatgttttactcagatacaggggagcaaagttccatttgctaaagtttactcccctgaccttcaaccagtcagtcttcctccatctgccaccactttgcacttctccagagaactaaggatgttcccgcttgaccagtgctcataacatggacagcagagggccactgtgtgatctctttgagatcactgtgactcaaccttcttctcacatcctaggccctaaaacaattaagtgaagttgctaggaacggtacctgctgatcttattgcagcattctcaattaggcctcaatgcaagatttatatcactggcagtcctggagcatttttgtttttcaaattacacatacccaaacacacggcatagcctccttttttgtttgtttgtttttttgagatagagtctcgctgtgtcgcccaggctggagtgcagtggcacgatctcagctcactgcaacctctgcctcctgggttcaagtgattctcatgtctcagcctcccaagtagctgagattacaggcgtataccaccacgcccagctaatttttgtatttttagtagagacagggttttgccgtgttggccaagctggtctcaaactcctgacctcaagtgatccacccacctcggcctcccgaagtgctgggattacaggtgtgagccaccgtgcccagccagggcatatccttcttgatttcaattgtaaaatagttcaaaaattttccatattttatctaatatttccagaagtgctagcttttaacggaccatttttttcctctgtgtgtttttttctcttcacctagcccagccatgctcagctcatttttgtactctttccactcccaaccaaatttagtgccctcccccatacatgcatacatgtacatctgcacaccacttttcctgcaaataatcaacccaaagagtgcttaaaattcctgacatcaacccacagaatctccaaggatgggacccagcatccatacattttaaaaactctccatatagttccaatatgcagccagatttgagaactagtggttcgtagcctgttctgatttaaatctcagctctcagcagtctatcccacgtcacataatgcagcccagagaaattctaggaccacatttttttctggtatttcatagctaatgaggtgcttttcaaatctaataggatctttggccagtgtcagtcaagatcttttatctcctcaataaaaaggaaataccatatttactttgatttgatgtatatcacataggtggatttaatacaaaattgtggtttacatattgtgaatgtgtatactaaaactactttgctttttcctaaaataagacaaagttttatattggaagtaatatttagcattttgtttgaatgaagttactcctattaaattagaaatttaaaagagggtcagtaataacagtaaagccaaaaggcatgacactgccaacgtaacataagctgctctgaaatctaccatatcaaaagataattatgctgggcatggtggctcacacctgtaatcccagcactttgggaggccaaggcaagagaattgcttgaagccaggagttcgagaccagcctgggaaatataatgataccttgcctcaaacaaaaattcaaaaattagccagcagtggtggcacacttgtaaaaatgcctgtagtcatagctacttcagaggctgagatgaaaggattgcttgggccaaggagttcgagactgcactccaacctgggaaatattgtgccactgcactccaacctgggaaacagaacaagaccctgtctctaaaataaaaagaaaaaaaaagatgaccacttctgaaatgacacctatcaatgagttaatcattcaatgaatatgtattgagtccctactatatgcttaggaacctttgtaatatcattaccaaccatgtctttcccaatacagacaatacaaaattcagcaataaataatatagcaccaacaattagagaataagacaacatgtagtatggtccaatatagacagtaaatacaaagacactgaataatatcagtaaaagtaaattcacatcaaggtcactacaccatgcgcccacccttatgatagccctcactggccctatcaattaagcaagagacatgatacaactctgtgcaagcttttccacaatctgcctaccattcagcactcagtcgctcttcccttcaattaagagaattgagcattcaagcatattttcaccatgatgcccataatggtatcttcaatgtcactgactgataaattcccagaaacccctcagagccccagccatgttagctcaaagcctttagctaaaactgaaagcctaaagcaaaagcagccctggctgcacttcggaatctactggacagctctttaagggattctgatttaatgtctggaatagggccaagaaccttgtattattttaaaggctcactagtaggctctaatatttagccgtggttgagaaccactgtgctaaatgtttcttaaatatgctttgtgatgtcatcataaattatattttagtattttttgtctttgttgcataagtgttctttcttcctccaaagaagaatgttacactcatttcttatttcagtttcctgttttcatagcacctcatcttaacactccaggctattatatagaaaagaatcaaatgtggagaaggctgtgggagaagggatgcctgtgccacaaaggcctgcattaggctgacctattgatgtcatatccaggactcaaaagactagtctgtggattatgactggtgaagttcaaaatgttcttattcttagagtggtatgagaagtagaaagagagagaaacagagaaggggaggagaggggaagagaggaagatgagagaaaggaaagagagggggaaacacctgttcttgacatacaggaatgattcaagacattttcttcctcccctgatgtgtccctttctcccctaacgcactatgcagcatcctgcagaaaattcaccacctgacccttttagaaaccctgagtagtaggagcgccaaatgacccaatcaagaattgcagtgagacagttagttttgaaaaatcagttaaagcatgtataatcattttaacaacaatacatctattcactaaacatataattttaatgtcaaatatttacgtgtaaacatattgaccaatctttcgatgtagttgggcccaataccttttccaaaaattgatcagttaatgggggttctatgggggtttcttttcttgccattattcacacttatgtcacattagctatgatttgcagttttaatttctttaaaattgagtagggactaaagacatctccaaaaagcctggatatagactttttacaacttttccatagcttttatagttgactcacccagtatctactaaatacttcactttctcacgtatttccaaaggtttctctccaccctcacaattttccattaatgtagtacttaattaaattagatagttaaattttcaaatgtgaattgctaaacaggtgtggaaataccattggctataatcaagcatataacacaaccatttgagaaggaaagtatgtggcaatattagggaagagccctttcctctcaagcaattcagcatttaggaaccatcagacagcaggacgatggagggaacagagagggttaacatggcaagttactgaagaggacttctactgaatcttgttgaattccccacttaatccagattgtatcatatcttctttcttttgtaattctaccatatcatcttagtcaatgccaagacttctgagctcataacatggtaacaaataccaaaggagctttcagtatcgtttagaaaggagagaagcaagtaacccagacaaacttgacaactgctttcccctatccaaccatgaagtacagtacttaggaaataaaagaaattgcttcactataattcatcatttcacttctaatatctagaaaatgtcaaatgaaaatattatagccatattttagtggcaatagtagcacataatatgatgcaacttaaaatgataaaaatattttcagggaataagattctgtgattctttccctaagaggtaattttgataatatgtacctgttttgtaaatgtcaatagtcttggggatacaggtggtgtttggttacatggaaaagttccttagtggtgatttctgagattttagtgcacccaatacccaagcagtgtacactgtacccaatatgtagtctttcatccctcgcccccactcccaaccttcccccacaagtctctaaagtccattatatcactcttatatctttgcatactcatagcttagctcccacttatgagaacatatgatagttagtgctcaattcctgagttacttcacttagaataatggcctccagctccacccaagttgctgcaaaagacactatttagttcctttttatggctgagtagtattacatggtgtatatataccacattttatttatccacttgttggtcaatggacacttaacattagttccatatctttgtaatttcaagttgtgctgctataagcatgcatgagcctgtgtctttttcatataattacttcttttcctttgggtagatacccagcagtgggattgctggatcaaatgatagttctactttcagttctttatgttttccacagtggtcatactaatttacattcccatcaacagtgtaaagtgttcccttttcatcacacccatgccaacacctattgttttttgactttttaattacggccattcttgcaggagtaaggtggtatttcattgtggttttaatttgcatttccctgatgttgacaatatttaactctttagttatagattccagctattatcaatttacacctattgcattcttctcatcttttgttttcttgtgattctgatgcacaaatatcatttgtgcaaccacttactgttgaacatgtctgatgaacacttactattgaacatgtctgatgaatgaataatgaaataggaaaagggattaaaactagcctttattaattgtttgctataggccagacatttttggatgtactatcacatttcatccaaacaacaacctaaaagaaaatactgtgattatccccatttcacatctaaggaacctggtctttaggaagattaagtcatttggccaagatcacaagtagaccacagagactagatttgaatgcaagtctgtttgactccaaacctttttactatctgcccatgacccctgatcaccaacatctcaatgtatgaacatgtgctttcttagctcacacaactcactcctgaccccttttttatattgcaagtgcatagtcattagtaaaaagaaggatttttgatgatactgacctcatcttgaatttaattaggctcatatgacagaattccatagatggaattgacatcctaggtcatatagtccaagtccttgtttatatttgatacctagtgagattaaagggacattaaaaagtaaagaaaggaaagacctcatatttcttaccttccagtagagaaatctttctatgaaatcagaggaaagaattagaggaccagaatttttcctaaaatcaactttcatacatcttttttcatataaaaggcatagctgcatacaatgctaaaatattgtattacatttcctttatattgatgggaggaagggggtaaattgcagaaaacattgtaaatttagatatgcttgggcctctgacagtgcctagcaaatatcaggagatcaataatgaaataaatattatcaaagagtagtcttcttgatgaaccttctctgagtatcacaactgctttaggaacctctagattcaaggtctagtaattgcaaacagtgagctgataagaaaaacagactgtatgggaaattacatgcttcctgcatgactgccttttgttctcccacattttgatataaagtcacattaacagttcatgagtaaatattcgataatgtgaacgtaaagtgttcaaataatagagtgactaaaatgcctgaaaacaaataatttttaattagaaactcataatcatttattttctctttttccacattatctcaagctcacaaattatatttattctttcctatggcaaaatccattttgttaacactaattttgagtttaacaagaagtgtactccaaagtagcctaataatactaattataatgtttcctgctatgttatcagtttgaatttatatgaatctttagacttgaggcttctttttcctagcatagtgatggtctgggctttttctcaatttttgccagagctcagctctcactaattagtttctttctgcatgagaaaaagattttgcttcatctttttccttataatagcagaacaaaaagaagaatcagctgcatccatgctaatttcccctgtgacatttccaaacaggatttgatttctctatgcatgcctctttccttctcttcatggtttttgaacatatacaaaagctcatttaaaccaattaaataaaattgtttttaatctctttctctagagtcaacttcctgcttactccaactctgtatctttgaaggaagtatagggtggtctatgccttttttctcccagaatctacacttgaaaagacacatttttccatgcaactataaaatgttctcctcactcaacattgaaattgtatagcagtgattaagagagtgagctgtagagccaggttccctgggtttaaatcccacttgttagtatcatgaagatgggcaagttacttacccttcctgtgtttcagtttcttcatctgcaaaatggggacaataatagaatgtccactataagattattgtgaggattaagggaattaatacaggtaaaacgtgtactgatgcaggtctggtacacattaagtgcctaataaatattcagtattatgatataaagaaccctataagtgtagactccttgagattaatagagtttaacgataagttttactttatagctggtcaagtttatttcttctgaactaaaagaatctatagagtctcaatttctggagcttcagagggaaggagagaagcaatgtaagcaacattctacagaaatataaataatactactaataattagcatcttaaaatttcaattcaatgaacatttatttagcgcctatgatatatgcaagacagtttgattttagtcatctgatgtatagccacatactaaaaaatactgattttagtcatctgatgtatagccacatactaaaaaatacttcctccatcagttccctcctcaggaagttcagttcccaatcccaggctagtaccttggttccttatgtaaataaacatccaccaattacatgctatctgcaaagcactctgctaggccctgcaaatggaaaaaaaaatgataaaacatagtccaggccctcaatgagcttacagtcaaatataatagaggagacaagaacagagaggctcataatacaactagaataaaatgactgccgaataaaaggaaagatttatgcaggtgttcaaatggaaagtgagataagtttgcaggttagtctttgcagtctcataaaaatctttatggagaaaaggacaatggtcatagggcttaaagagtaagtttataatcctgaccagtggagatgaaagactagcattgaaaattgcatgacaagacaattccattaaactgaaacatcaagtgtgtgtaggaaaagatgggggttatgactggaaacgtcacttggactgcaattatgaagggccttgacaaacaggtcaagagtttaagaagcagtatagaaagtcttcgtcctggatctagccctcccagagtgtccatcaggattataaagtccttaaaatattagtcaaaaggaacgacatcattagaaatgatagagaaacaataatgtgatgttttattacctttctctggatttatactctgatcctaatattcaaaactatcttaataacatgaacttttggtcatagttttaaacaaaaacagtgttaaatatattttttaaaacacagtaagtcttgtaagatcttttctaacatgacattttgcagggcccatattttccttctgaaatgggaaaaattcataaaagtagacaccaaactgggttacttctagtcaagcgcatggtacgcaaaggaccagacaaaaagggcctgtgacatttcttcttccttttgtgttttttaggagaggcatgctgcggaggtgcgcaggaacaaggaactccaggttgaactgtctggctgaagcaagggagggtctggcacgccccaccaatagtaaatccccctgcctatattataatggatcatgcgatatcaggatggggaatgtatgacatggtttaaaaagaactcattataaaaaaaaaaaaacaaaaaaaatcaaaaattaaaaaaaatcaatgcggtctctttgcagaatgttttgcttgatgtttaaaaaataccttggatcttattttgtaaatacttacatttttgttaaaaaatacaagtattgcattatgcaagttatttcataatcttacatgtcctgtaacaggcttttgatgttgtgtctttccactcaaatgaatttgctaggtctgttctttttgaagctccccatgtctaactccattccaaaagaaaaatgaggtcagtagacagtctatggtgctagaaacccaccattgcctaatgacctagaaggctttgttgtctctgagcttgactaagaccatacctagatcacaggtattatgactccacatgaaccttcacatttgttcgctcataatctacttactgcctaaaaactacaaaaccaggctaagaaataccaccagtcatagcatttacttctgcttctcctggattatgtgctacaaatgtgctttggctttagaaagggatggatgagaagacagacctgagaccaatctgggtagaagcaaaaagttgaaccttttaaagtgctgaacacaaatccaaattcgaatggttcaagcagccgtgaaatcgctcttcataaagtgggcttaattctctagtttaagttcttttgatggaatgaattaattaatgtgtcaggtggcttatttgtggatgccatgattgatgatgttcattttaagctcttacctatagtacaagtacatgatgctactgaatatttttccacttggaaactgtgagctggttgttgcattaaaacacacatacaaacaaaatcaaaaacactgcggactttcactcaagctggtctttcttccccagtgtaaggcaatcctgcctactaacaacaccaacaacaaaacactccatctgtgaagctgacgcagttaagggggctaggcagggcatttgtgccaactaagaatcaccagatacccaccataagtacctatcgcagttttgaagtcgtttctccccaactcccaactcctgaaggttgctgcctgcatatttactcttcattagtgctattttcctgtatgtcattgtgagcaagctgtgattaataaagaattggagttctgtgaactaataaa

SEQ ID NO: 2 - DNA, 21 bp, Artificial Sequence
  Synthetic
cctgtccctggaggagatcca

SEQ ID NO: 3 - DNA, 22 bp, Artificial Sequence
  Synthetic
catgttggcatggcacaggttc

SEQ ID NO: 4 - DNA, 21 bp, Artificial Sequence
  Synthetic
cttctctctcgctctctccgc

SEQ ID NO: 5 - DNA, 25 bp, Artificial Sequence
  Synthetic
cttgtaggccattgctgttttagcc

SEQ ID NO: 6 - DNA, 20 bp, Artificial Sequence
  Synthetic
gaagatggtgatgggatttc

SEQ ID NO: 7 - DNA, 19 bp, Artificial Sequence
  Synthetic
gaaggtgaaggtcggagtc

SEQ ID NO: 8 - DNA, 26 bp, Artificial Sequence
  Synthetic
cttatggtgcaggtcaagaaagatct

SEQ ID NO: 9 - DNA, 25 bp, Artificial Sequence
  Synthetic
cgctgtgacattactttcacttgtg

SEQ ID NO: 10 - DNA, 21 bp, Artificial Sequence
  Synthetic
cttctctctcgctctctccgc

SEQ ID NO: 11 - DNA, 25 bp, Artificial Sequence
  Synthetic
cttgtaggccattgctgttttagcc

(19) United States
(12) Patent Application Publication
(10) Pub. No.: US 2023/0348977 A1
(43) Pub. Date: Nov. 2, 2023

(54) A BIOMARKER AND USES THEREFOR

(71) Applicants: The University of Western Australia, Crawley (AU); Murdoch University, Rockingham (AU)

(72) Inventors: Frances THEUNISSEN, Ascot (AU); Patrick Anthony AKKARI, Mullaloo (AU); Loren FLYNN, Karrinyup (AU); Ryan ANDERTON, North Beach (AU)

(73) Assignees: The University of Western Australia, Crawley (AU); Murdoch University, Rockingham (AU)

(21) Appl. No.: 17/782,314

(22) PCT Filed: Dec. 4, 2020
(86) PCT No.: PCT/AU2020/051330

§ 371 Date: Jun. 3, 2022

(30) Foreign Application Priority Data
Dec. 4, 2019 (AU) 2019904595

(51) Int. Cl. C12Q 1/6883

(52) U.S. Cl. CPC C12Q 1/6883; C12Q 2600/112; C12Q 2600/118; C12Q 2600/156

(57) ABSTRACT

The present disclosure relates generally to methods and protocols for the diagnosis, prognosis and stratification of patients with, or at risk of developing, sporadic amyotrophic lateral sclerosis (ALS). In particular, the methods and protocols of the present disclosure are based on determination of the presence and number of cytosine (C) adenine (A) dinucleotide repeats (CA dinucleotides) within the STMN2 gene.

### FIELD OF THE ART

The present disclosure relates generally to methods and protocols for the diagnosis, prognosis and stratification of patients with, or at risk of developing, sporadic amyotrophic lateral sclerosis (ALS). In particular, the methods and protocols of the present disclosure are based on determination of the presence and number of cytosine (C) adenine (A) dinucleotide repeats (CA dinucleotides) within the STMN2 gene.

### RELATED APPLICATIONS

This application claims priority from Australian Provisional Patent Application No. 2019904595 filed on 6 Dec. 2019, the entire content of which is hereby incorporated by reference.

### BACKGROUND

Amyotrophic lateral sclerosis (ALS), also known as Lou Gehrig’s disease, is a progressive neurodegenerative disorder characterized by the loss of lower and upper motor neurons, resulting in paralysis and respiratory failure. ALS is characterised by rapid disease progression, with 50% of patients dying within 3 years of disease onset. There are currently no curative treatments available for patients with ALS, with the standard of care largely comprising symptomatic relief, with the use of supportive treatments including physiotherapy, occupational therapy, speech therapy, maintenance of respiratory function, nutritional therapy and physiological support shown to improve patient survival and quality of life (Miller et al., 2009, Neurology, 73: 1227-1233). Pharmacological agents such as riluzole are also used for the treatment of ALS, which extends survival of patients by a modest 3-6 months (Bensimon et al., 1994, New England Journal of Medicine, 330: 585-591; Lacomblez et al., 1996, The Lancet, 347: 1425-1431). More recently, the neuroprotective agent edaravone has also been approved for use in the treatment of ALS following clinical studies indicating that patients treated with edaravone show a significantly smaller decline in ALS Functional Rating Scale (ALSFRS-R) score as compared to placebo. However, the positive clinical outcomes associated with edaravone appears to be limited to a small subset of ALS patients and there is no indication that edaravone is effective for the treatment of ALS more generally (The Writing Group et al., 2017, The Lancet Neurology, 16(7): 505-512).

The lack of progress in the development of new pharmacological interventions to treat or delay the progression of the ALS is reflective of the lack of understanding of pathogenesis and genetic basis of the disease. It is widely recognised that ALS is a complex disease, with very little known about the precise mechanisms involved in the pathogenesis of the disease. The majority of ALS cases are classified as sporadic ALS (~90%), with the remaining 10% of cases being inherited (familial ALS). While some progress has been made to define genes and variants that are associated with ALS or a predisposition to develop ALS, only 11% of sporadic ALS cases can be explained by mutations in known ALS genes, such as SOD1, FUS, TARDP, OPTN, VCP, PFN1, UBQLN2 and C9orf72 (Renton et al., 2014, Nature Neuroscience, 17(1): 17). Accordingly, there is a need to identify genetic factors predictive of a state of, or genetic predisposition to developing sporadic ALS. Such genetic factors further provide targets for therapeutic intervention.

### SUMMARY OF THE DISCLOSURE

The present disclosure is predicated, in part, on the surprising finding that the presence of two long alleles of the STMN2 gene, wherein at least one of the long alleles comprises ≥ 24 consecutive cytosine (C) adenine (A) dinucleotides (CA dinucleotides) provides an indication that a subject has, or has a genetic predisposition to develop, sporadic amyotrophic lateral sclerosis (ALS). Further, it has also been shown that the number of consecutive CA dinucleotides present in each allele of the STMN2 gene is predictive of key clinical indicators including age of onset, rate of disease progression and survival. These findings have been reduced to practice in a method for diagnosing sporadic ALS in a human subject, a method for determining the outcome of disease in a subject with sporadic ALS, a method for predicting the age of onset in a subject with a genetic predisposition to develop sporadic ALS and methods for the treatment of sporadic ALS.

Accordingly, in one aspect, the present disclosure provides a method for determining if a subject has, or has a genetic predisposition to develop, sporadic amyotrophic lateral sclerosis (ALS), the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of cytosine (C) adenine (A) dinucleotides (CA dinucleotides) from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, wherein long alleles have ≥ 19 consecutive CA dinucleotides in the STMN2 gene,
wherein the identification of two long alleles (L/L genotype) of the STMN2 gene, wherein at least one of the long alleles comprises ≥ 24 consecutive CA dinucleotides, is indicative that the subject has, or has a genetic predisposition to develop, sporadic ALS.

In another aspect, the present disclosure provides a method for predicting the outcome of disease in a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1; and
d. identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥ 19 consecutive CA dinucleotides in the STMN2 gene.

In another aspect, the present disclosure provides a method for predicting the age of onset in a subject with a genetic predisposition to develop sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1; and
d. identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥ 19 consecutive CA dinucleotides in the STMN2 gene,
and wherein a subject with at least one long allele (L/L or S/L genotype) has an earlier predicted age of onset as compared to a subject with two short alleles (S/S genotype).

In another aspect, the present disclosure provides a method for the treatment of a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, wherein long alleles have ≥ 19 consecutive CA dinucleotides in the STMN2 gene,
e. identifying the subject as having sporadic ALS, wherein the presence of two long alleles (L/L genotype) of the STMN2 gene, wherein at least one of the long alleles comprises ≥ 24 consecutive CA dinucleotides, is indicative that the subject has sporadic ALS; and
f. treating a subject identified as having sporadic ALS in (d) with a treatment for said sporadic ALS.

In another aspect, the present disclosure provides a method for the treatment of a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have <19 consecutive CA dinucleotides in the STMN2 gene, wherein long alleles have ≥19 consecutive CA dinucleotides in the STMN2 gene;
e. stratifying the subject for treatment of said ALS based on the presence of at least one short allele (S/L or S/S genotype) or the presence of two long alleles (L/L genotype); and
f. treating the subject stratified in (e) with a treatment for said sporadic ALS.

In another aspect, the present disclosure provides a kit for determining if a subject has, or has a genetic predisposition to developing, sporadic ALS, the kit comprising a probe or set of oligonucleotides designed to determining the nucleotide sequence of a fragment of genomic DNA comprising at least a portion of the nucleotide sequence of the STMN2 gene set forth in SEQ ID NO: 1.

### BRIEF DESCRIPTION OF THE DRAWINGS

Embodiments of the disclosure are described herein, by way of non-limiting example only, with reference to the accompanying drawings.

FIG. 1 is a graphical representation of allele length (CA repeat length; x-axis) of the STMN2 gene and frequency (y-axis) in cases (left panel) and controls (right panel). Samples with ≥19 consecutive CA dinucleotides were considered to have a long (L) allele and samples with <19 consecutive CA dinucleotides were considered to have a short (S) allele.

FIG. 2 is a graphical representation of allele length (CA repeat length; x-axis) of the STMN2 gene and frequency (y-axis) in Coreill University cases (upper panels) and Duke University cases (lower panels). Samples with ≥19 consecutive CA dinucleotides were considered to have a long (L) allele and samples with <19 consecutive CA dinucleotides were considered to have a short (S) allele.

FIG. 3 is a graphical representation of the distribution of age of onset (years; y-axis) in cases with a short allele (S/S genotype) and with at least one long allele (S/L or L/L genotype).

FIG. 4 is a graphical representation of cumulative survival (y-axis) and duration (years; x-axis) in sporadic ALS patients with at least one short allele (S/S and S/L genotype; grey line) and two long alleles (L/L genotype; black line) of STMN2. Kaplan Meier analysis of cumulative survival, p = 0.2.

FIG. 5 is a graphical representation of STMN2 gene expression (relative mRNA expression; y-axis) in patients and controls with at least one short allele (S/L genotype; Other) and patients with two long alleles (L/L genotype).

FIG. 6 is a schematic representation of the STMN2 primary transcript encoding 179 amino acids.

FIG. 7 is a graphical representation of is a graphical representation of cumulative survival (y-axis) and duration (years; x-axis) in sporadic ALS patients stratified by CA genotype and site of disease onset. Kaplan Meier analysis of cumulative survival, p = 0.006.

FIG. 8 is a photographic representation of STMN2 and TARDBP gene expression in olfactory neurosphere derived (ONS) cells with at least one short allele (S/L genotype) and two long alleles (L/L genotype) from sporadic ALS patients (lanes 5-8) and control ONS cell lines (lanes 1-4).

FIG. 9 is a graphical representation of relative STMN2 and TARDBP gene expression (y-axis) as measured by densitometry standardized to GAPDH gene expression.

FIG. 10 is a graphical representation of STMN2 gene expression (y-axis) in laser captured spinal motor neurons with at least one short allele (S/L genotype) and two long alleles (L/L genotype) from sporadic ALS patients and controls (x-axis). Error bars represent standard error of the mean.

FIG. 11 is a graphical representation of (A) the percentage of motor neurons positive for phosphorylated TDP-43 according to the STMN2 CA genotype; and (B) motor neuron death according to STMN2 CA genotype.

Nucleic acid sequences are referred to by a sequence identified number (SEQ ID NO). Sequences are provided in the Sequence Listing.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a treatment” includes a single treatment, as well as two or more treatments; and so forth.

In the context of this specification, the term “about” is understood to refer to a range of numbers that a person of skill in the art would consider equivalent to the recited value in the context of achieving the same function or result.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

Currently, there are few genetic markers that explain incidence, genetic predisposition, variability in progression, survival and age of onset relating to sporadic amyotrophic lateral sclerosis (ALS). For example, the presence of at least one minor G allele of ST3GAL3 indicates a two-year difference in the age of onset (Alsgen et al. 2013, Neurobiology of Aging, 34(1): 357) and presence of the SLC11A2 C allele in lower limb onset patients, exhibited decreased survival of 17.5 months (Blasco et al., 2011, Journal of Neurological Sciences, 303(1): 124-127). However, only 11% of sporadic ALS cases can be explained by mutations or variants in known ALS genes. Identification of further genetic markers that explain the remaining incidence, genetic predisposition, and clinical outcomes associated with sporadic ALS is essential in developing new pharmacological interventions for the treatment of sporadic ALS. As described herein, a novel genetic marker for determining if a subject has, or has a genetic predisposition to develop, sporadic ALS has been identified. In particular, the presence of two long alleles of the STMN2 gene, wherein at least one of the long alleles comprises ≥ 24 consecutive cytosine (C) adenine (A) dinucleotides (CA dinucleotides), provides an indication that a subject has, or has a genetic predisposition to develop, sporadic ALS. A distinct advantage of the genetic marker of the present disclosure is that structural variation in the length of the alleles or the presence of at least one short allele is predictive of the outcome of disease (i.e., rate of progression, survival) in patients with sporadic ALS and the age of onset for subjects with a genetic predisposition to develop sporadic ALS. The genetic marker described herein is also useful in the stratification of patients for treatment of sporadic ALS and associated methods of treatment.

In an aspect, the present disclosure provides a method for determining if a subject has, or has a genetic predisposition to develop, sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥ 19 consecutive CA dinucleotides in the STMN2 gene,
and wherein the identification of two long alleles (L/L genotype) of the STMN2 gene, wherein at least one of the long alleles comprises ≥ 24 consecutive CA dinucleotides, is indicative that the subject has, or has a genetic predisposition to develop, sporadic ALS.

### Amyotrophic Lateral Sclerosis (ALS)

The terms “amyotrophic lateral sclerosis” or “ALS” as used herein refers to progressive motor neuron disease. The disease is characterised by degeneration of motor neurons in the cortex, brain stem and spinal cord. In ALS, the neurons of the cerebral cortex and anterior horns of the spinal cord, together with their homologues in the motor nuclei of the brain stem, are affected. Mortality due to ALS is typically the result or respiratory failure secondary to profound generalised and diaphragmatic weakness.

ALS may be inherited as an autosomal dominant trait (i.e., familial ALS) or occur sporadically (i.e., sporadic ALS).

In an exemplary embodiment, the ALS is sporadic ALS.

### STMN2

The term “STMN2 gene” as used herein refers to a gene encoding a phosphoprotein that is a member of the stathmin family of proteins. Stathmin proteins have role in microtubule dynamics, cytoskeletal arrangement and signal transduction in cells. The Stathmin 2 protein has been proposed as an axonal maintenance factor that can accelerate neurodegeneration when expression is depleted, making this gene highly relevant in the context of ALS (Klim et al., 2019, Nature Neuroscience, 22: 167-179). Reduced expression of STMN2 has also been associated with Alzheimer’s disease (Okazaki et al., 1995, Neurobiology of Aging, 16(6): 883-894), Down’s syndrome (Bahn et al., 2002, The Lancet, 359(9303): 310-315) and spinal muscular atrophy (Wen et al., 2010, Human Molecular Genetics, 19(9): 1766-1778). While STMN2 knockdown in human motor neurons effects neurite growth and these cells fail to exhibit extension even in the presence of ROCK inhibitor, which significantly increased the outgrowth of wild type STMN2 cell lines (Klim et al., 2019, supra).

The term “gene” as used herein refers to one or more sequence(s) of nucleotides in a genome that together encode one or more expressed molecules, e.g., an RNA, or polypeptide. The gene can include coding sequences that are transcribed into RNA, which may then be translated into a polypeptide sequence, and can include associated structural or regulatory sequences that aid in replication or expression of the gene.

As used herein the terms “polynucleotide”, “nucleotide sequence” or “nucleic acid sequence” mean a single- or double-stranded polymer of deoxyribonucleotide, ribonucleotide bases or known analogues or natural nucleotides, or mixtures thereof.

In an embodiment, the STMN2 gene is encoded by the nucleotide sequence set forth in SEQ ID NO: 1.

### Structural Variants

The term “structural variant” as used herein refers to highly polymorphic small structural variants. Structural variants provide local association data at the structural variant locus, for example, allelic variation in the length and composition of structural variants can differentiate between specific phenotypic risks in a population such as age of onset related to variable length polymorphisms and risk of phenotypic variations associated with several adjacent structural variants. Suitable structural variants will be known to persons skilled in the art, illustrative examples of which include variable length Simple Sequence Repeats (e.g., SSRs, STRs or microsatellites) as described by Roses et al. (2016, Expert Opinion on Drug Metabolism and Toxicology, 12(2): 135-147).

In an embodiment, the structural variant is a CA dinucleotide repeat comprising two or more consecutive CA dinucleotides. Consecutive CA dinucleotides may be interchangeably referred to herein as “CA repeats”, “CA dinucleotide repeats” and “CA repeat sequences.”

In an embodiment, the number of consecutive CA dinucleotides is between 2 and 50 consecutive CA dinucleotides. The phrase “between 2 and 50 consecutive CA dinucleotides” as used herein means 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 CA dinucleotides.

In a preferred embodiment, the number of consecutive CA dinucleotides is between 10 and 26 consecutive CA dinucleotides.

The person skilled in the art would appreciate that the phrase “consecutive CA dinucleotides” may be alternatively referred to as “CA repeats”. It follows, therefore, that embodiments disclosed herein may encompass between 2 and 50 CA repeats, and so on.

The presence of structural variants in the STMN2 gene may result in the variant alleles of the STMN2 gene that define a genotype associated with having, or having a genetic predisposition to develop sporadic ALS.

The term “genotype” as used herein refers to the genetic constitution of an individual (or group of individuals) at one or more genetic loci. Genotype is defined by the allele(s) of one or more known loci of the individual, typically, the compilation of alleles inherited from its parents.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a SNP, which is a polymorphism at a single nucleotide position in a genome (the nucleotide at the specified position varies between individuals or populations).

The term “short allele” as used herein refers to an allele comprising < 19 consecutive CA dinucleotides in the STMN2 gene.

The term “long allele” as used herein refers to an allele comprising ≥ 19 consecutive CA dinucleotides in the STMN2 gene.

In an embodiment, at least one long allele comprises ≥ 24 consecutive CA dinucleotides.

In another embodiment, at least one long allele comprises 24 consecutive CA dinucleotides.

In an embodiment, a subject with two long alleles of the STMN2 gene may be referred to as having an L/L genotype.

In an embodiment, a subject with two short alleles of the STMN2 gene may be referred to as having an S/S genotype.

In another embodiment, a subject with a long allele and a short allele of the STMN2 gene may be referred to as having an S/L or L/S genotype.

The term “allele frequency” as used herein refers to the frequency (proportion or percentage) at which an allele is present at a locus within an individual, within a line, or within a population of lines. For example, for an allele “A” diploid individuals of genotype “AA”, “Aa” or “aa” may have allele frequencies of 2, 1, or 0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “locus” as used herein refers to a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

As exemplified herein, data representing structural variants may be obtained by in silico analysis of variants in published datasets (e.g., NCBI and Ensembl Genome Browser). Structural variants identified in any such in silico analysis may be confirmed by determining the nucleotide sequence of a fragment of genomic DNA sample and detecting in the nucleotide sequence the presence or absence of structural variants.

In an embodiment, a sample comprising genomic DNA is obtained from the subject.

Genomic DNA may be obtained from any sample of suitable biological source material containing genomic DNA. Suitable biological source materials would be known to persons skilled in the art, illustrative examples of which include tissue, saliva or blood obtained from the subject. The biological source material may be in a form taken directly from the subject, or may be at least partially processed (purified) to remove at least some non-nucleic acid material.

In an embodiment, the sample is a blood sample.

In an embodiment, the nucleotide sequence of a fragment of the genomic DNA comprising at least a portion of the nucleotide sequence of the STMN2 gene set forth in SEQ ID NO: 1 is determined following amplification of the genomic DNA.

The term “amplification” as used herein means any conventional means of generating sufficient nucleic acid material for analysis. Suitable methods for the amplification of nucleic acids would be known to persons skilled in the art, illustrative examples including polymerase chain reaction (PCR) methods, as exemplified herein.

In an embodiment, the genomic DNA is amplified using oligonucleotide primers having the nucleotide sequences set forth in SEQ ID NO: 2 and 3, or functional fragments thereof.

The nucleotide sequence of amplified DNA may then be determined using nucleic acid sequencing technology that would be known to persons skilled in the art, illustrative examples of which include Sanger sequencing and next-generation sequencing (also referred to as sequencing by synthesis).

It is contemplated herein that the disclosed methods may be used in conjunction with existing clinical criteria for the diagnosis of sporadic ALS. Sporadic ALS is currently diagnosed by reference to the El Escorial World Federation of Neurology Criteria for Diagnosis of ALS (Brooks, 1994, Journal of the Neurological Sciences, 303(1): 124-127).

Accordingly, in an embodiment, the subject has one or more clinical symptoms of ALS, wherein the clinical symptoms of ALS are selected from the group consisting of lower motor neuron (LMN) degeneration, upper motor neuron (UMN) degeneration, and combinations thereof.

Methods for the assessment of LMN degeneration would be known to persons skilled in the art, illustrative examples of which include clinical examination of weakness, wasting and fasciculation, electrophysiological examination and neuropathological examination in one or more of the four regions relevant to the diagnosis of ALS (i.e., bulbar, cervical, thoracic, and lumbosacral).

Methods for the assessment of UMN degeneration would be known to persons skilled in the art, illustrative examples of which include clinical examination of increased or donic tendon reflexes, spasticity, pseudo bulbar features, Hoffman reflex and extensor plantar response in one or more of the four regions relevant to the diagnosis of ALS as described elsewhere herein. The bulbar, cervical and lumbosacral regions are of particular relevance to an assessment of UMN degeneration.

In an embodiment, the clinical symptoms of ALS may also be accompanied by one or more secondary clinical symptoms, wherein the secondary clinical symptoms are selected from the group consisting of abnormal pulmonary function, abnormal speech, abnormal swallowing, abnormal larynx function, abnormal isokinetic or isometric strength, abnormal muscle biopsy with evidence of denervation, and combinations thereof.

A person skilled in the art would appreciate that the secondary clinical symptoms described herein are only of relevance to the diagnosis of ALS when the abnormal clinical symptoms are not explained by other causes and where the subject also exhibits one or more of the clinical symptoms of ALS and/or has at least two long alleles (L/L genotype) of the STMN2 gene.

It has been exemplified herein that the long allele of the STMN2 gene, comprising ≥ 19 consecutive CA dinucleotides, more frequently occurs in subjects with ALS as compared to controls. Accordingly, in an embodiment, the presence of a long allele with 24 consecutive CA dinucleotides indicates that a subject has, or has a genetic predisposition to develop, sporadic ALS. In another embodiment, in subjects with two long alleles, the risk associated with having or developing sporadic ALS is increased where at least one of the long alleles comprises ≥ 24 consecutive CA dinucleotides.

### Methods for Predicting Disease Outcome

In another aspect, the present disclosure provides a method for predicting the outcome of disease in a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1; and
identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥ 19 consecutive CA dinucleotides in the STMN2 gene.

In an embodiment, the outcome of disease is selected from the group consisting of rate of progression, survival rate, and combinations thereof.

As exemplified herein, the presence of two long alleles (L/L genotype) of the STMN2 gene is predictive of a faster rate of disease progression and poorer survival outcomes as compared with the presence of at least one short allele (S/S or S/L genotype).

In an embodiment, the outcome of disease is rate of progression, wherein a subject with two long alleles (L/L genotype) has a faster rate of progression as compared to a subject with at least one short allele (S/S or S/L genotype).

In another embodiment, the outcome of disease is survival rate, wherein a subject with two long alleles (L/L genotype) has a poor survival rate as compared to a subject with at least one short allele (S/S or S/L genotype).

### Methods for Predicting Age of Onset

In another aspect, the present disclosure provides a method for predicting the age of onset in a subject with a genetic predisposition to develop sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1; and
d. identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥ 19 consecutive CA dinucleotides in the STMN2 gene,
and wherein a subject with at least one long allele (L/L or S/L genotype) has an earlier predicted age of onset as compared to a subject with two short alleles (S/S genotype).

As exemplified herein, the presence of at least one long allele (L/L or S/L genotype) of the STMN2 gene is predictive of an earlier age of onset of sporadic ALS in subjects with a genetic predisposition to develop sporadic ALS as compared to a subject with two short alleles (S/S genotype) of the STMN2 gene.

In an embodiment, a subject with at least one long allele (L/L or S/L genotype) of the STMN2 gene has a predicted age of onset that is between about 5 and 10 years earlier than a subject with two short alleles (S/S genotype). The phrase “between 5 and 10 years” as used herein means 5, 6, 7, 8, 9, or 10 years.

In an exemplary embodiment, a subject with at least one long allele (L/L or S/L genotype) of the STMN2 gene has a predicted age of onset that is about 7.5 years earlier than a subject with two short alleles (S/S genotype).

### Methods for the Treatment of Sporadic ALS

In another aspect, the present disclosure provides a method for the treatment of a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of cytosine CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, wherein long alleles have ≥ 19 consecutive CA dinucleotides in the STMN2 gene,
e. identifying the subject as having sporadic ALS, wherein the presence of two long alleles (L/L genotype) of the STMN2 gene, wherein at least one of the long alleles comprises ≥ 24 consecutive CA dinucleotides, is indicative that the subject has sporadic ALS; and
f. treating a subject identified as having sporadic ALS in (d) with a treatment for said sporadic ALS.

In another aspect, the present disclosure provides a method for the treatment of a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have <19 consecutive CA dinucleotides in the STMN2 gene, wherein long alleles have ≥19 consecutive CA dinucleotides in the STMN2 gene;
e. stratifying the subject for treatment of said ALS based on the presence of at least one short allele (S/L or S/S genotype) or the presence of two long alleles (L/L genotype); and
f. treating the subject stratified in (e) with a treatment for said sporadic ALS.

As used herein the terms “treat”, “treating”, “treatment”, “prevent”, “preventing”, “prevention”, “prophylaxis” and the like refer to any and all methods that remedy, prevent, hinder, retard, ameliorate, reduce, delay or reverse the progression of ALS or one or more undesirable symptoms thereof in any way. Thus, the terms “treating” and “preventing” and the like are to be considered in their broadest context. For example, treatment does not necessarily imply that a patient is treated until total recovery. Sporadic ALS is characterised by multiple symptoms, and thus the treatment need not necessarily remedy, prevent, hinder, retard, ameliorate, reduce, delay or reverse all of said symptoms. Methods of the present disclosure may involve “treating” a sporadic ALS in terms of reducing or ameliorating the occurrence of a highly undesirable event or symptom associated with sporadic ALS or an outcome of the progression of the disorder, but may not of itself prevent the initial occurrence of the event, symptom or outcome. Accordingly, treatment includes amelioration of the symptoms of sporadic ALS or preventing or otherwise reducing the risk of developing symptoms of sporadic ALS.

As exemplified herein the genotype of a subject with sporadic ALS influences the functional status of sporadic ALS patients. In particular, subjects with two long alleles (L/L genotype) of the STMN2 gene exhibit a more rapid functional decline as compared to subjects with at least one short allele (S/S or S/L genotype).

Functional decline according to genotype may be assessed using clinical scales that would be known to persons skilled in the art, illustrative examples of which include the Amyotrophic Lateral Sclerosis Functional Rating Scale (ALSFRS), which is used to monitor functional change in an ALS patient over time, as described by ALS CNTF Treatment Study (ACTS) Phase I-II Study Group (1996, Archives of Neurology, 53: 141-147) and Cedarbaum et al. (1997, Journal of Neurological Sciences, 152 (Supplementary 1): S1-S9).

In an embodiment, the treatment for sporadic ALS is selected from the group consisting of riluzole, edaravone, medications for symptomatic relief, physical therapy, nutritional therapy and combinations thereof.

Suitable medications for symptomatic relief would be known to persons skilled in the art, illustrative examples of which include any therapeutic agent that reduces or alleviates one or more of the clinical symptoms associated with ALS such as morphine, benzodiazepines, baclofen, botulinum toxin, gabapentin, opioid drugs, antidepressant agents, and glycopyrrolate.

As used herein, the term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, it will be understood by persons skilled in the art that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” also includes a cell which is capable of producing and secreting the agents referred to herein, as well as a polynucleotide comprising a nucleotide sequence that encode such agents. Thus, the term “agent” extends to nucleic acid constructs including vectors such as viral or non-viral vectors, expression vectors and plasmids for expression in and secretion in a range of cells.

The therapeutic regimen for the prevention of sporadic ALS will typically depend on factors including, but not limited to, the stage and extent of the disease and the age, weight, and general health of the subject. Another determinative factor will be the predicted outcomes of disease determined by the methods disclosed herein. For instance, for a subject identified as having a genetic predisposition to develop sporadic ALS, a preventative therapeutic regimen may be prescribed to increase the age of onset as compared to a subject deemed to have sporadic ALS with predicted rapid disease progression and poor survival, where a more aggressive therapeutic regimen may be prescribed to extend the survival rate.

It is further contemplated that the detection of the genetic marker described herein is useful in the stratification of patients to a particular therapeutic regimen with a higher likelihood of treatment benefit. For example, it has previously been demonstrated that the treatment benefit of lithium carbonate, creatine and valproic acid was only observed by stratifying ALS patients for these treatments based on the presence of the UNC13A (C/C) genotype (van Eijk et al., 2017, Neurology, 89(18): 1915-1922; van Eijk et al., 2019, The Pharmacogenomics Journal, 1-7). Accordingly, it is demonstrated that therapeutic response in ALS patients is at least partially determined by patient genotype. On this basis, it is reasonable to expect that the novel genetic marker described herein may also be useful in reducing cohort heterogeneity and identifying ALS patients who will response to treatments as a result of the L/L, L/S or S/S STMN2 genotype.

In an embodiment, use of the genetic marker described herein in the stratification of patients to a particular therapeutic regimen may be combined with one or more markers of ALS pathophysiology. Suitable markers of ALS pathophysiology would be known to persons skilled in the art, illustrative examples of which include site of disease onset (e.g., spinal, bulbar).

Accordingly, in an embodiment, the detection of the genetic marker described herein and the site of disease onset is useful in the stratification of patients to a particular therapeutic regimen with a higher likelihood of treatment benefit.

As exemplified herein, the genotype of subject with sporadic ALS influences basal STMN2 gene expression. In particular, the number of consecutive CA dinucleotides may play a functional role in determining the levels of STMN2 expression. A person skilled in the art would appreciate, therefore, the subjects may also be stratified to a particular therapeutic regimen based on an assessment of basal STMN2 gene expression, which provides a further genotypic marker that is dependent on the number of consecutive CA dinucleotides in the STMN2 gene.

### Kits

In another aspect, the present disclosure provides a kit for determining if a subject has, or has a genetic predisposition to developing, sporadic ALS, the kit comprising a probe or set of oligonucleotides designed to determining the nucleotide sequence of a fragment of genomic DNA comprising at least a portion of the nucleotide sequence of the STMN2 gene set forth in SEQ ID NO: 1.

In an embodiment, the kit comprises oligonucleotide primers comprising the nucleotide sequences set forth in SEQ ID NO: 2 and 3, or functional fragments thereof.

Such kits may also include suitable software, or access to suitable software, to facilitate identification and comparisons between long allele and short allele frequencies, and to facilitate statistical analysis that may be employed for such functions.

Kits for carrying out the methods of the present disclosure may include, suitable container means comprising, or adapted to receive, reagents required. The container means may include at least one vial, test tube, flask, bottle, syringe and/or other container. The kits may also include means for containing the reagents in close confinement for commercial sale.

Kits may also include suitable means to receive a genomic DNA sample, one or more containers or vessels for carrying out methods described herein, positive and negative controls, including a reference sample, and instructions for the use of kit components contained therein, in accordance with the methods disclosed herein.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### General Methods

### Structural Variant (SV) Selection

Structural variants (SVs) likely to modulate expression regulation within known ALS genes were identified, evaluated and prioritised using the SV evaluation algorithm described by Saul et al. (2016, Human Mutation, 37(9): 877-883). Candidate variants were scored according to 24 different properties, including variability (e.g., number, size and simple sequence repeat slippage), synergy of consecutive variants, trait association, nearby regulatory elements, signal for transcription factor binding sites, region conserved among mammals and primates, local drop in conservation value and intron size.

### Polymerase Chain Reaction

PCR reactions were prepared to a final volume of 10 µl, containing; 7.2 µl dH₂O (Baxter Healthcare, NSW, Australia), 2 µl MyFi reaction buffer (Bioline,), 0.05 µl Myfi DNA polymerase (Bioline), 0.5 µM forward primer (SEQ ID NO: 2; 5′-CCTGTCCCTGGAGGAGATCCA -3′), 0.5 µM reverse primer (SEQ ID NO: 3; 5′-CATGTTGGCATGGCACAGGTTC-3′), and 10 ng genomic DNA. Amplification was performed according to the following protocol: an initial hold temperature 95° C. for 4 min 30 sec, 35 cycles of denaturation at 95° C. for 30 sec, annealing at 56° C. for 30 sec and extension at 72° C. for 1 min. PCR products were subsequently confirmed by electrophoresis on a 2% agarose gel.

### Polyacrylamide Fractionation

PCR products were fractionated on 8% (w/v) 29:1 polyacrylamide gel in 1× TBE. Electrophoresis fragment separation was performed at 100 V for 16.5 hr on the DCode™ Universal Mutation Detection System (Biorad). Gels were stained in 1× TBE containing SYBR® Gold nucleic acid gel stain (Thermo Fisher Scientific) for 4 mins before visualisation using a BioRad Chemidoc™ MP Imaging System.

### Confirmation of Allele Length

In heterozygous cases, individual amplicons were purified by band-stab and sequenced. Gels were visualised on a BluPAD GeneDireX-BP001CU transilluminator and each band of interest was excised with a pipette tip before dispersion into a 30 µl pre-prepared PCR mix and re-amplified. Band stabbed PCR amplicons were confirmed by electrophoresis on a 2% agarose gel before purification and Sanger sequencing. Analysis was conducted using Finch-TV software (version 1.5.0; Geospiza Inc.).

### High-Throughput Genotyping

Due to the variability of genomic DNA fragments comprising consecutive CA dinucleotides, genotyping was performed by capillary separation using fluorescent end labelled primers to improve genotype resolution. PCR reaction were carried out using the above method with a FAM-labelled forward primer.

### Sporadic ALS Cohort

321 Caucasian North American ALS patients and 332 Caucasian North American age-matched controls were used in this study. Genomic DNA samples were collected from patients and controls for genotyping and analysis. Genomic DNA samples from 176 patients were provided by Duke University Neurology Clinic and the remaining patient and control samples were obtained from Coriell Institute for Medical Research. ALS patients included in the cohort were diagnosed by board-certified neurologists and met the revised El Escorial World Federation of Neurology Criteria for Diagnosis of ALS (Brooks, 1994, supra).

### Statistics

Case-control binary genotype associations were assessed using a Pearson’s Chi-squared test, while case-control logistic regression was used to assess joint genotypic effects. General linear models were used for the prediction of age of onset by genotype category. Within the SALSA longitudinal study, patient clinical characteristics were assessed over time using general linear mixed models (LMMs). Naïve LMMs were utilised to assess whether patient clinical characteristics were significantly associated with ALSFRS, over-time. Variables assessed included age at symptom onset, time between follow up, age at assessment, and site of disease onset. Corrected LMMs were constructed to assess the impact STMN2 genotype group (L/L) on ALSFRS, independently of covariates. Variables identified as being statistically significant were included in the multivariable corrected LMMs. Residual plots were examined for all models and no violations were noted. Survival times were assessed using Kaplan-Meir analysis and Cox proportional hazards models. A p-value below 0.05 was considered significant. Analyses were carried out in IBM SPSS Statistics version 25.0 (IBM Co., Armonk, NY, USA).

### Olfactory Neurosphere Derived Cells

Olfactory neurosphere derived cells (ONS) were derived from olfactory mucosal cells extracted from both sporadic ALS patients and healthy controls in accordance with the methods described by Murrell et al. (2005, Developmental Dynamics: an Official Publication of the American Association of Anatomists, 233(2): 496-515). The resulting ONS cells were genotyped for the long and short CA alleles and mRNA expression analysed using the methods described elsewhere herein.

### Reverse Transcription-Polymerase Chain Reaction

RT-PCR reactions were prepared to a final volume of 12.5 µl, containing: 3.9 µl dH₂O (Baxter Healthcare, NSW, Australia), 6.25 µl 2× Reaction mix buffer (ThermoFisher Scientific) 0.35 µl platinum Taq (ThermoFisher Scientific), 0.1 µM forward primer (SEQ ID NO: 4; 5′- CTTCTCTCTCGCTCTCTCCGC-3′), 0.1 µM reverse primer (SEQ ID NO: 5; 5′-CTTGTAGGCCATTGCTGTTTTAGCC -3′), and 1 µl of 10 ng RNA. Amplification was performed according to the following protocol: a single reverse transcription step was carried out at 55° C. for 30 min followed by 94° C. for 2 min. Amplification steps were carried out as follows: 40 cycles of denaturation at 94° C. for 40 sec, annealing at 58° C. for 30 sec and extension at 68° C. for 1 min. PCR products were subsequently confirmed by electrophoresis on a 2% agarose gel. Densitometry was conducted using ImageJ 1.25A (National Institute of Health, USA) to quantitate levels of STMN2 mRNA for patients and controls with the S/L and L/L genotype, standardising against levels of reference gene GAPDH.

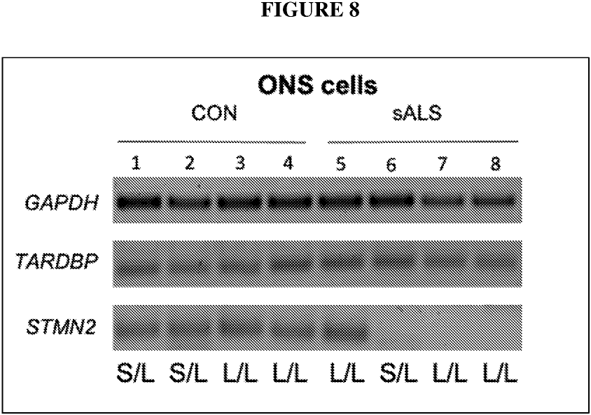

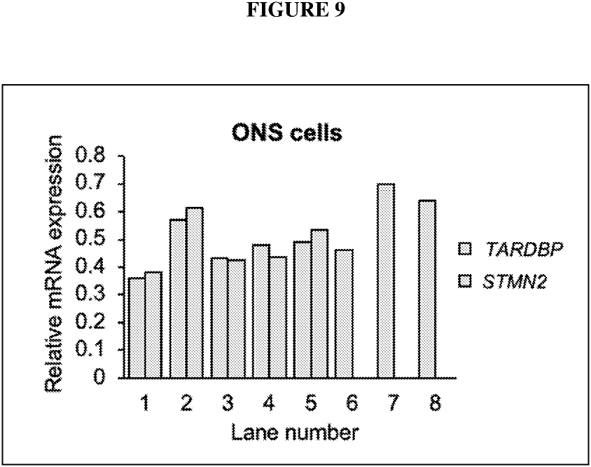

For the gene expression analyses of ONS presented in FIGS. 8 and 9, RNA was extracted using TRIzol (Thermo Fisher Scientific, MA, USA) as per manufacturer’s instructions and resuspended in 30 µl of dH₂O (Baxter Healthcare, NSW, Australia). cDNA was synthesized using the SuperScript IV system (Thermo Fisher Scientific, MA, USA) in 20 µl reactions as per manufacturer’s instructions, using 10 µl of template RNA with a total of 500 ng. Endpoint PCR reactions for GAPDH and TARDBP were prepared to a final volume of 10 µl, containing; 7.2 µl of dH₂O (Baxter Healthcare, NSW, Australia), 2 µl of MyFi reaction buffer (Bioline, NSW, Australia), 0.05 µl of MyFi DNA polymerase (Bioline, NSW, Australia), 0.375 µl of GAPDH (SEQ ID NO: 6; 5′-GAAGATGGTGATGGGATTTC-3′) or TARDBP (SEQ ID NO: 8; 5′-CTTATGGTGCAGGTCAAGAAAGATCT-3′) forward primer and, 0.375 µl of GAPDH (SEQ ID NO: 7; 5′-GAAGGTGAAGGTCGGAGTC-3′) or TARDBP (SEQ ID NO: 9; 5′-CGCTGTGACATTACTTTCACTTGTG-3′) reverse primer, at 200 ng/µl, and 25 ng of cDNA. STMN2 reactions amplifying across exons 1-2 were prepared to a final volume of 10 µl, containing; 7.2 µl of dH₂O (Baxter Healthcare, NSW, Australia), 2 µl of GC reaction buffer (Thermo Fisher Scientific, MA, USA), 0.01 µl of hot start Phusion polymerase (Thermo Fisher Scientific, MA, USA), 0.375 µl of forward (SEQ ID NO: 10; 5′-CTTCTCTCTCGCTCTCTCCGC-3′) and, 0.375 µl of reverse (SEQ ID NO: 11; 5′-CTTGTAGGCCATTGCTGTTTTAGCC-3′) primer at 200 ng/µl, and 25 ng of cDNA. The amplification protocols for GAPDH and TARDBP followed an initial hold temperature of 95° C. for 4 min 30 sec, and 24 (GAPDH) and 35 (TARDBP) cycles of denaturation 95° C. for 30 sec, annealing 57° C. (GAPDH) or 60° C. (TARDBP) for 30 sec and extension 72° C. for 1 min. The amplification of STMN2 followed an initial hold temperature of 98° C. for 30 sec followed by 40 cycles of denaturation 98° C. for 10 sec, annealing 57° C. for 30 sec and extension at 72° C. for 15 sec. PCR products were then fractionated on a 2% agarose (w/v) gel (Scientifix Pty Ltd, VIC, Australia) and stained with red safe nucleic acid stain (iNtRON Biotechnology, Scientifix Pty Ltd, VIC, Australia) prior to being image captured using the BioRad Chemidoc™ MP Imaging System, followed by densitometry using image J software (National Institutes of Health, MD, USA).

### Laser Capture Microdissection (LCM) and RNA Extraction

Laser capture microdissection of spinal motor neurons was performed as described in Rabin et al. (2010, Human Molecules Genetics, 19: 313-328). RNA isolation was performed using the RNeasy Micro kit (Qiagen) in accordance with the manufacturer’s instructions. Aliquots of each RNA pool were analysed for quality and quantity using a Bioanalyzer RNA Pico chip (Agilent) and only those samples with RNA Integrity Numbers (RINs) > 5 or with evidence of 28S peaks on the electropherogram traces were used for RNA sequencing (RNA-seq). Following RNA isolation, DNA was collected from laser captured spinal motor neurons for STMN2 CA genotyping.

### RNA-Seq Library Preparation and Sequencing

Total RNA (10 ng) was amplified to cDNA using random priming with the Ovation RNA-Seq System (NuGEN) in accordance with the manufacturer’s instructions. The single-stranded cDNA was then copied into double-stranded cDNA and quantified using the PicoGreen kit (Invitrogen) assayed with the FUSION system (Packard Biosciences), resulting in a total yield of 3-4 ug amplified cDNAs. Standard concentration curves using bacteriophage lambda DNA were generated for each PicoGreen analysis, and the samples were diluted by ten-fold serial dilutions. QC of the amplified cDNA was determined using a Bioanalyzer running an RNA 6000 Nano LabChip (Agilent). Double-stranded cDNA (1-2 µg) was fragmented to 150-200 bp sizes using Adaptive Focused Acoustics™ (Covaris, Inc., Woburn, MA), and QC of the fragmented cDNA was performed using a Bioanalyzer DNA Chip 1000 (Agilent). Fragmented cDNAs were concentrated using the QIAquick PCR Purification Kit (Qiagen) and 200 ng of the fragmented cDNAs were end-repaired, followed by adaptor ligation onto the fragments and amplification using the Encore® NGS Library System I (NuGEN). Library QC was performed using a Bioanalyzer DNA Chip 1000.

### Perth SALSA Cohort

67 sporadic ALS patients from the Perth SALSA longitudinal cohort were used to investigate survival and disease progression. Patients were genotyped using genomic DNA extracted from blood. All participants were diagnosed by board-certified neurologists and met the revised El Escorial World Federation of Neurology criteria for diagnosis of ALS (Brooks, 1994, supra).

Sporadic ALS patients were assessed at multiple time points using the ALS function rating score (ALSFRS) respective of disease duration. Follow-up times varied for patients and appointments were organised based on clinical neurologist recommendations. On average 4 follow-up visits were recorded or each of the 67 patients.

### Example 1 - Identification of Polymorphic Structural Variant in the STMN2 Gene

A structural variant in the STMN2 gene was identified in silico using the SV evaluation algorithm described by Saul et al. (2016, supra). The identified variant comprised consecutive CA dinucleotides (i.e., a CA repeat region), with STMN2 gene sequences of varying lengths and with varying numbers of consecutive CA dinucleotides recorded in NCBI and Ensembl Genome Browser databases. However, no allele frequency data or disease associations were available for this variant.

The variant was identified and confirmed in genomic DNA samples from of 321 ALS patients and 332 controls. Initially, the variant was assessed on conventional polyacrylamide gel electrophoresis and 6 alleles were identified with 15, 19, 20, 21, 22 and 23 consecutive CA dinucleotides, as confirmed by Sanger sequencing. To increase the resolution of detection, an alternative genotyping method using capillary separation with a fluorescent end labelled primer was also used to identify variant alleles. This alternative genotypic method identified 8 additional alleles with 10, 11, 13, 17, 18, 24, 25 and 26 consecutive CA dinucleotides, as confirmed by Sanger sequencing.

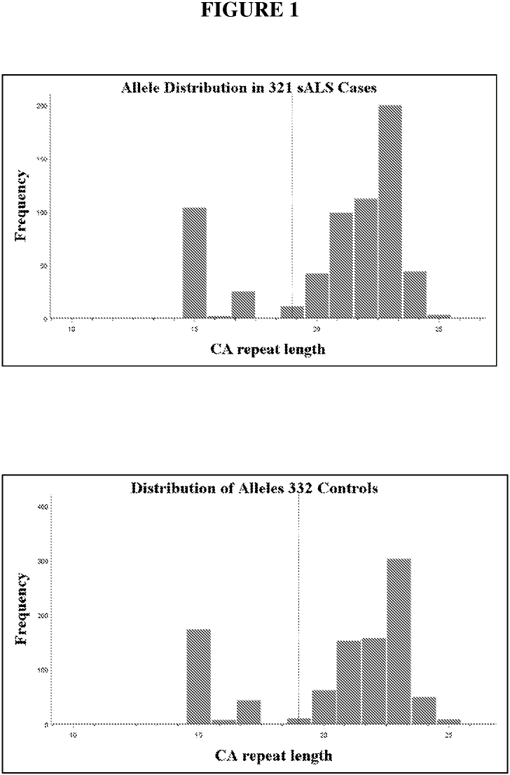

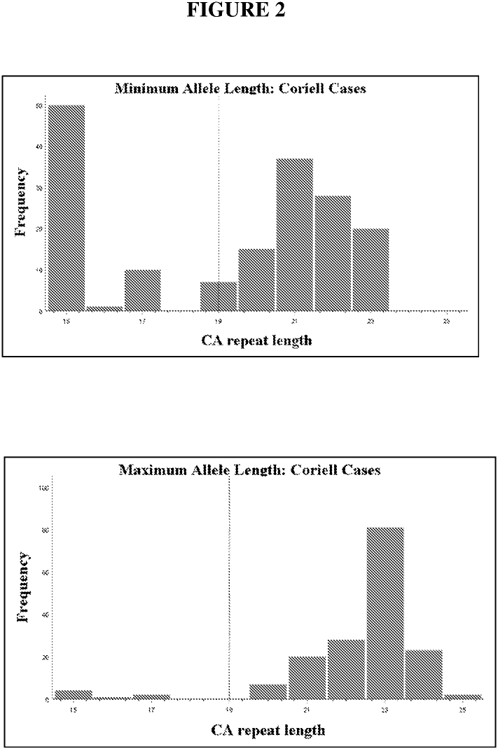

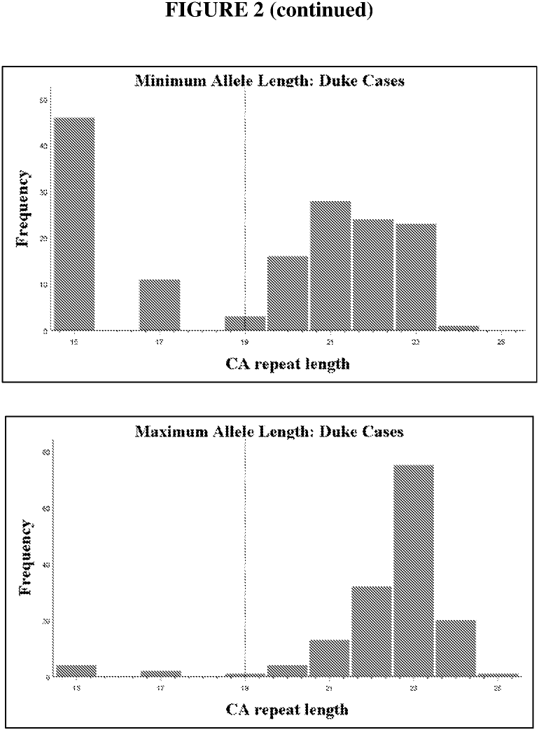

For both cases and controls, the distribution of alleles separated the cohort into two groups based on allele length: alleles with <19 consecutive CA dinucleotides (i.e., short alleles) and alleles with ≥19 consecutive CA dinucleotides (i.e., long alleles), based on the distribution of alleles (FIG. 1). Details of allele length (i.e., genotype), together with cohort characteristics including gender and age are provided in Table 1. The distribution of alleles for the Duke and Coriell samples were analysed separately (FIG. 2). Both patient groups consisted of individuals with self-reported Caucasian ethnicity. Country of origin information was not available for all cases however, to determine whether cases could be appropriately combined, we compared the relative allele distributions and did not see a significant difference between the Coriell and Duke cases (p > 0.8). Each distribution was also compared to distributions on Webstr database and were similar to both Gtex (predominantly European self-reported ancestry) and 1000 genomes European allele distributions, giving us confidence that the population in this study is reflective of a Caucasian population with European descent. Therefore, the two patient groups were combined to increase the sample size and statistical power to detect genetic effects.

There was a significant difference in the frequency of the long allele between cases (63%) and controls (55%) (χ²= 4.12, p = 0.042, odds ratio (OR) = 1.4, 95% confidence interval (CI) = 1.02-1.92), with more cases having two copies of the long alleles (≥ 19CA) as compared to controls.

TABLE 1

Cohort characteristics for sALS patients and controls

| Variable | | Patients n = 321 | Controls n = 332 | Total |
|---|---|---|---|---|
| Gender | Male | 174 | 167 | 341 |
| | Female | 147 | 165 | 312 |
| Age | | 58.01 (av. 13.09) | 56.61 (av. 15.38) | |
| Two long alleles | L/L | 203 | 183 | 386 |
| Long/short | L/S | 105 | 135 | 240 |
| Short/short | S/S | 13 | 14 | 27 |

### Example 2 - Long Alleles of the STMN2 Gene Are Associated With Sporadic ALS

The frequency of the 24CA variant was also examined in the cohort of 321 ALS patients and 332 controls (Table 2). The presence of 24CA repeat was associated with cases (χ² = 4.19, p = 0.041, OR = 1.75, CI = 1.05 - 2.90,). To consider the impact of having two long alleles (≥19CA) with the presence or absence of the 24CA repeat, a case-control logistic regression was performed. There was an increased frequency in cases of those carrying two long alleles with at least one being 24CA repeat (p = 0.0023, adjusted OR = 2.6, CI = 1.41 - 4.97), whilst those carrying two long alleles not carrying a 24CA repeat allele did not differ significantly between cases and controls (p = 0.15, adjusted OR = 1.27, CI = 0.92 - 1.76). This effect was not abrogated by age or gender (Table 3).

TABLE 2

Cohort characteristics for L/L genotype sALS patients and controls

| Variable | n (%) | | |
|---|---|---|---|
| | Patients n = 203 | Controls = 183 | Total |
| At least one 24CA | 37 (11.5%) | 18 (5.4%) | 55 |
| No 24CA | 166 (51.5%) | 165 (49.7%) | 331 |

TABLE 3

Case-control logistic regression for the association of cases/controls with the presence of two long alleles (≥19CA) and at least one 24CA allele

| | Estimate | Standard error | P-value | P-value^{#} |
|---|---|---|---|---|
| Long allele and 24CA repeat | 0.95 | 0.31 | 0.0023 | 0.017 |
| Long allele no 24CA repeat | 0.24 | 0.17 | 0.15 | 0.15 |
| Age | - | - | - | 0.17 |
| Gender | - | - | - | 0.35 |

^{#}Model including age and gender.

### Example 3 - Long Alleles of the STMN2 Gene Are Associated With Age of Onset and Disease Outcome

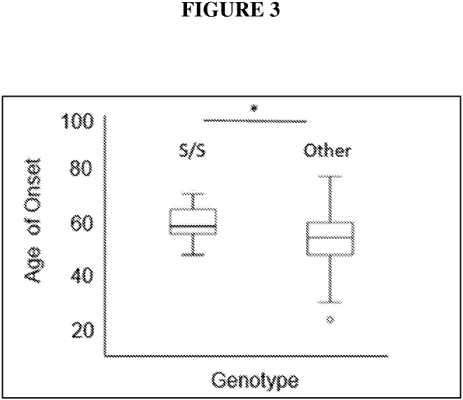

The presence of at least one long allele (≥19CA) was associated with a significantly lower age at onset in sporadic ALS patients (FIG. 3), with an estimated mean difference of approximately 7.5 years (p = 0.039, CI = 0.43 - 14.97).

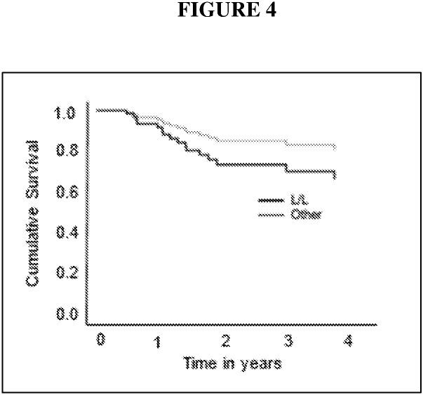

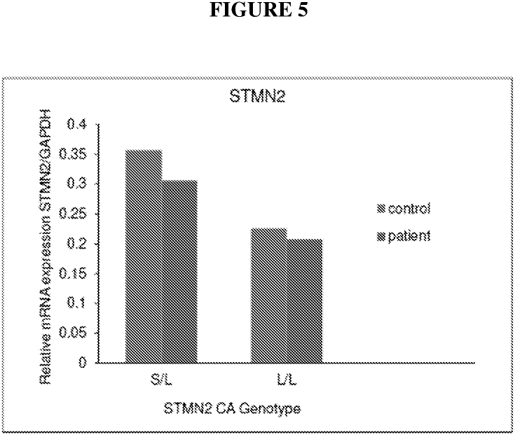

A distinct separation between patients with two long alleles (L/L genotype) as compared to patients with one long allele (S/L genotype) was observed in a Kaplan Meier analysis of cumulative survival (FIG. 4). These data indicate that the presence of two long alleles is detrimental to patient survival outcomes.

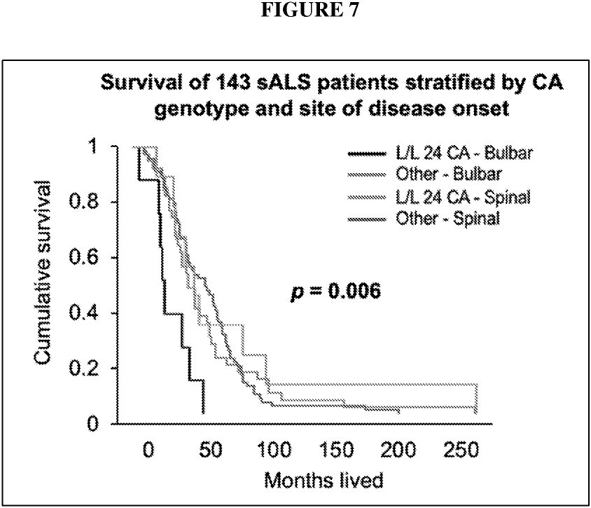

When the covariate of site of onset was taken into account, there was a significant decrease in survival for bulbar onset patients with the risk genotypes (L/L and 24CA) compared to bulbar patients with other genotypes and spinal onset patients without the L/L and 24CA genotypes (p = 0.006, hazard ratio (HR) = 3.4, CI = 1.6 - 1.7) (FIG. 7).

A generalised linear mixed model was to predict the impact of the presence of two long alleles (L/L genotype) as compared to patients with at least one short allele (S/S or S/L genotype) in the Perth SALSA longitudinal ALS patient cohort. The model included significant predictors of change in ALSFRS. The final model investigating CA genotype group (L/L vs other) included age at onset of symptoms, time between follow ups, age at assessment, and site of disease onset as covariates. When accounting for repeated measures, analyses revealed that the CA variant was significantly associated with ALSFRS score, with the L/L genotype accounting for a decrease in 2.37 ALSFRS points. The estimated means derived from the GLMM revealed the L/L genotype to be significantly lower (28.98) than participants with other genotypes (S/S or S/L; 31.35) (p = 0.034; Table 4).

TABLE 4

General linear mixed model with predictors of ALSFRS score over time in Caucasian Australian sALS patients (SALSA cohort)

| Model Term | Coefficient | Std Error | t | p | 95% CI | |
|---|---|---|---|---|---|---|
| | | | | | Lower | Upper |
| Intercept | 34.30 | 3.46 | 9.91 | 0.000 | 27.49 | 41.11 |
| Age at symptom onset | 0.53 | 0.17 | 3.13 | 0.002 | 0.19 | 0.85 |
| Follow up time (mo) | -0.18 | 0.06 | -3.13 | 0.002 | -0.29 | -0.07 |
| Age (yr) | -0.59 | 0.16 | -3.69 | 0.000 | -0.90 | -0.27 |
| Spinal onset | 5.22 | 1.27 | 4.12 | 0.000 | 2.72 | 7.72 |
| Bulbar onset | 0^{b} | · | · | · | · | · |
| Other genotypes | 2.37 | 1.12 | 2.14 | 0.034 | 0.18 | 4.55 |
| L/L | 0^{b} | · | · | · | · | · |

### Example 4 - Long Alleles of the STMN2 Gene Are Associated With Lower Basal Expression of STMN2 mRNA

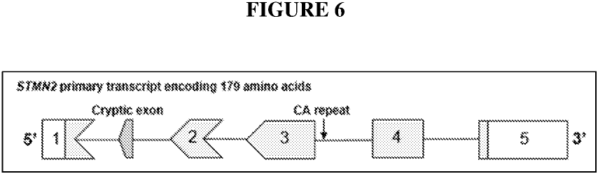

Relative gene expression was assessed in mRNA isolated from neurosphere-derived cells obtained from sporadic ALS patients and healthy controls (FIG. 6). When taking into account the presence of two long alleles (L/L genotype) or the presence of one short allele (S/L genotype), the S/L genotype was shown to have higher basal expression of STMN2 as compared to the L/L genotype in both cases and controls. These data demonstrate that the number of consecutive CA repeats may play a functional role in determining levels of STMN2 expression.

The decrease in STMN2 expression associated with the L/L genotype was further validated in neurosphere-derived cells from an additional three sporadic ALS patients and healthy controls, when standardised to GAPDH, as compared with the expression of TARDBP standardised to GAPDH (FIGS. 8 and 9). These data demonstrate that there is no significant difference in TARDBP expression between cases and controls (p = 0.19). It was also shown that the two sporadic ALS cells lines that had no detectable STMN2 also carried longer versions of the CA repeat, with at least one allele with 24 consecutive CA dinucleotides.

### Example 5 - Long Alleles of the STMN2 Gene Are Associated With Lower Basal Expression of STMN2 mRNA in Laser Captured Spinal Motor Neurons

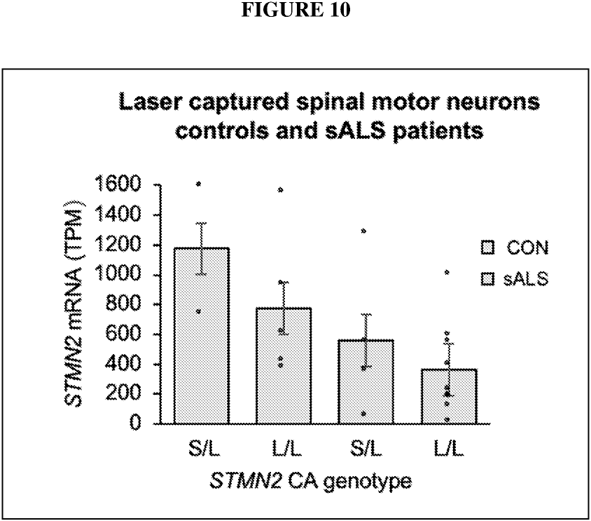

Expression of STMN2 was assessed in mRNA isolated from sporadic ALS patients and healthy controls (Table 5) in accordance with the methods described by Krach et al. (2018, Acta Neuropathologica, 136(3): 405-423) and STMN2 expression between cases and controls as previously described by Melamed et al. (2019, Nature Neuroscience, 22(2): 180. When taking into account the presence of two long alleles (L/L genotype) or the presence of one short allele (S/L genotype), the S/L genotype was shown to have higher basal expression of STMN2 as compared to the L/L genotype in both cases and controls (FIG. 10).

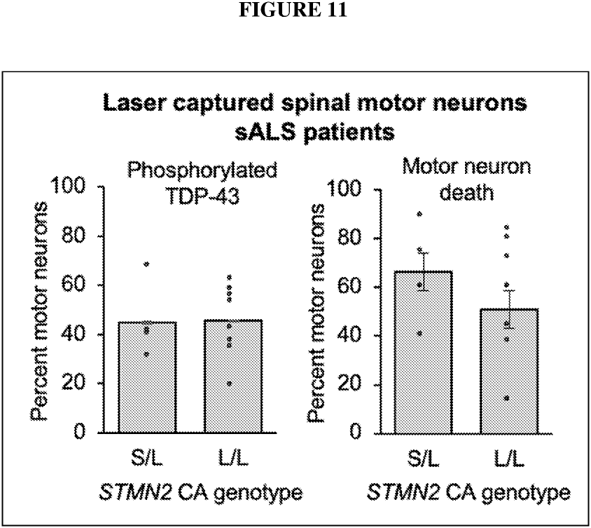

To confirm that the differential expression of STMN2 observed between cases with the S/L and L/L CA genotypes was not associated with differences in disease severity, the relative TDP-43 pathology in the spinal cords of the patients with the S/L or L/L CA genotypes was assessed. No difference was observed in the percentage of phosphorylated TDP-43 positive motor neurons between the two groups (FIG. 11). Additionally, no significant difference in the percentage of motor neuron death was observed between the two groups (FIG. 11). These data demonstrate that the differential expression of STMN2 observed between cases with the S/L and L/L CA genotypes was not associated with differences in disease severity.

TABLE 5

Cohort characteristics for laser captured motor neurons from sALS patients and controls

| | Gender | Age | Site of onset | Disease course (yr) | Cause of death | PMI | STMN2 (TPM) | STMN2 CA genotype |
|---|---|---|---|---|---|---|---|---|
| CONTRLS | M | 78 | NA | NA | Sepsis/pulmonary hypertension | 2.5 | 367 | 22/23 |
| | M | 77 | NA | NA | Aortic dissection/MSF | 2 | 925 | 23/23 |
| | F | 80 | NA | NA | Liver failure | 5 | 415 | 23/23 |
| | M | 82 | NA | NA | NA | 4 | 1600 | 17/21 |
| | M | 77 | NA | NA | NA | 4 | 606 | 21/21 |
| | M | 68 | NA | NA | Sepsis, ARDS, ARF | 4 | 751 | 15/23 |
| | F | 78 | NA | NA | CVA, MSF | 3.8 | 1552 | 21/21 |
| sAL,S | M | 61 | Arm | 2.5 | NA | 3.5 | 550 | 17/21 |
| | M | 84 | Respiratory and hand | 1.2 | NA | 2 | 996 | 21/23 |
| | M | 74 | Bulbar | 3.25 | NA | 4 | 359 | 17/21 |
| | F | 81 | Bulbar | 1 | NA | 3.5 | 22 | 20/21 |
| | M | 67 | Bulbar | 1.75 | NA | 6 | 217 | 21/23 |
| | F | 58 | Bulbar | 3 | NA | 3 | 558 | 21/22 |
| | M | 52 | Arm | 1.67 | NA | 6 | 590 | 23/23 |
| | M | 68 | Arm | 2.5 | NA | 5 | 399 | 21/22 |
| | M | 55 | Arm | NA | NA | 5 | 1274 | 15/24 |
| | M | 54 | Bulbar | 2.5 | NA | 8 | 192 | 21/23 |
| | F | 56 | Bulbar | 2 | NA | 4 | 192 | 21/22 |
| | F | 77 | Trunk | 2.3 | NA | NA | 120 | 22/23 |
| | M | 36 | Bulbar | 3 | NA | 5 | 187 | 15/21 |

*MSF multi system failure, ARDS acute respiratory distress syndrome, ARF acute renal failure, CVA cerebrovascular accident, PMI postmortem interval, TPM transcripts per million

Collectively, these data demonstrate that the presence of two long alleles of the STMN2 gene are associated with sporadic ALS and genetic predisposition to develop sporadic ALS (p = 0.042, OR = 1.4, CI = 1.02 - 1.92). In this context, the presence of at least one allele with 24 consecutive CA dinucleotides, in subjects with two long alleles has been demonstrated to be highly significant in distinguishing between cases and controls (p = 0.0023, OR = 2.6, CI = 1.41 - 4.97). The presence of at least one long allele (L/L or S/L genotype) has also been demonstrated to be predictive of the age of onset in subjects with a genetic predisposition to develop sporadic ALS (p = 0.039, CI = 0.43 - 14.7). The presence of two long alleles with one allele being 24 consecutive CA dinucleotides (L/L 24CA), is also associated with a significant reduction is survival in a subgroup of patients when accounting for initial site of disease onset p = 0.006, hazard ratio [HR] = 3.4, CI = 1.6 - 1.7). The presence of two long alleles (L/L) is also associated with a reduction in ALSFRS progression score over time compared to (S/S and S/L) groups (p = 0.034), with the number of consecutive CA dinucleotides associated with the predictive value of this genetic marker.

These data enable the use of the disclosed genetic variant as a biomarker for the diagnosis of sporadic ALS, the identification of subjects with a genetic predisposition to develop sporadic ALS, predicting disease outcomes in patients with sporadic ALS, predicting age of onset in subjects with a genetic predisposition to develop sporadic ALS and in methods for the treatment of sporadic ALS patients.

1. A method for determining if a subject has, or has a genetic predisposition to develop, sporadic amyotrophic lateral sclerosis (ALS), the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of cytosine (C) adenine (A) dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, wherein long alleles have ≥ 19 consecutive CA dinucleotides in the STMN2 gene,
wherein the identification of two long alleles (L/L genotype) of the STMN2 gene,
wherein at least one of the long alleles comprises ≥24 consecutive CA dinucleotides, is indicative that the subject has, or has a genetic predisposition to develop, sporadic ALS.

2. The method of claim 1, wherein at least one allele comprises 24 consecutive CA dinucleotides.

3. The method of claims 1 or claim 2, wherein the nucleotide sequence of (b) is determined following amplification of the genomic DNA.

4. The method of claim 3, wherein the genomic DNA is amplified using oligonucleotide primers having the nucleotide sequences set forth in SEQ ID NO: 2 and 3, or functional fragments thereof.

5. The method of any one of claims 1 to 4, wherein the sample is a blood sample.

6. The method of any one of claims 1 to 5, wherein the subject has one or more clinical symptoms of ALS, wherein the clinical symptoms of ALS are selected from the group consisting of lower motor neuron (LMN) degeneration, upper motor neuron (UMN) degeneration, and combinations thereof.

7. The method of claim 6, wherein the clinical symptoms of ALS are accompanied by one or more secondary clinical symptoms, wherein the secondary clinical symptoms are selected from the group consisting of abnormal pulmonary function, abnormal speech, abnormal swallowing, abnormal larynx function, abnormal isokinetic or isometric strength, abnormal muscle biopsy with evidence of denervation, and combinations thereof.

8. A method for predicting the outcome of disease in a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1; and
d. identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥19 consecutive CA dinucleotides in the STMN2 gene.

9. The method of claim 8, wherein the nucleotide sequence of (b) is determined following amplification of the genomic DNA.

10. The method of claim 9, wherein the genomic DNA is amplified using oligonucleotide primers having the nucleotide sequences set forth in SEQ ID NO: 2 and 3, or functional fragments thereof.

11. The method of any one of claims 8 to 10, wherein the sample is a blood sample.

12. The method of any one of claims 8 to 11, wherein the outcome of disease is selected from the group consisting of rate of progression, survival rate and combinations thereof.

13. The method of any one of claims 8 to 12, wherein the outcome of disease is rate of progression, and wherein a subject with two long alleles (L/L genotype) has a faster rate of progression as compared to a subject with at least one short allele (S/L or S/S genotype).

14. The method of any one of claims 8 to 13, wherein the outcome of disease is survival rate, and wherein a subject with two long alleles (L/L genotype) has a poor survival rate as compared to a subject with at least one short allele (S/L or S/S genotype).

15. A method for predicting the age of onset in a subject with a genetic predisposition to develop sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1; and
d. identifying short (S) alleles and long (L) alleles in nucleotide sequences with CA dinucleotides in the STMN2 gene detected in (c), wherein short alleles have < 19 consecutive CA dinucleotides in the STMN2 gene, and wherein long alleles have ≥19 consecutive CA dinucleotides in the STMN2 gene,
and wherein a subject with at least one long allele (L/L or S/L genotype) has an earlier predicted age of onset as compared to a subject with two short alleles (S/S genotype).

16. The method of claim 15, wherein the nucleotide sequence of (b) is determined following amplification of the genomic DNA.

17. The method of claim 16, wherein the genomic DNA is amplified using oligonucleotide primers having the nucleotide sequences set forth in SEQ ID NO: 2 and 3, or functional fragments thereof.

18. The method of any one of claims 15 to 17, wherein the sample is a blood sample.

19. The method of any one of claims 15 to 18, wherein a subject with at least one long allele (L/L or S/L genotype) has a predicted age of onset that is between 5 and 10 years earlier than a subject with two short alleles (S/S genotype).

20. The method of claim 19, wherein a subject with at least one long allele (L/L or S/L genotype) has a predicted age of onset that is about 7.5 years earlier than a subject with two short alleles (S/S genotype).

21. A method for the treatment of a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, wherein long alleles have ≥ 19 consecutive CA dinucleotides in the STMN2 gene,
e. identifying the subject as having sporadic ALS, wherein the presence of two long alleles (L/L genotype) of the STMN2 gene, wherein at least one of the long alleles comprises ≥24 consecutive CA dinucleotides, is indicative that the subject has sporadic ALS; and
f. treating a subject identified as having sporadic ALS in (d) with a treatment for said sporadic ALS.

22. The method of claim 21, wherein at least one of the long alleles comprises 24 consecutive CA dinucleotides.

23. The method of claim 21 or claim 22, wherein the nucleotide sequence of (b) is determined following amplification of the genomic DNA.

24. The method of claim 23, wherein the genomic DNA is amplified using oligonucleotides having the nucleotide sequences set forth in SEQ ID NO: 2 and 3, or functional fragments thereof.

25. The method of any one of claims 21 to 24, wherein the sample is a blood sample.

26. The method of any one of claims 21 to 25, wherein the subject has one or more clinical symptoms of ALS, wherein the clinical symptoms of ALS are selected from the group consisting of lower motor neuron (LMN) degeneration, upper motor neuron (UMN) degeneration, and combinations thereof.

27. The method of claim 26, wherein the clinical symptoms of ALS are accompanied by one or more secondary clinical symptoms, wherein the secondary clinical symptoms are selected from the group consisting of abnormal pulmonary function, abnormal speech, abnormal swallowing, abnormal larynx function, abnormal isokinetic or isometric strength, abnormal muscle biopsy with evidence of denervation, and combinations thereof.

28. The method of any one of claims 21 to 27, wherein the treatment for sporadic ALS is selected from the group consisting of riluzole, edaravone, medication for symptomatic relief, physical therapy, nutritional therapy and combinations thereof.

29. A method for the treatment of a subject with sporadic ALS, the method comprising:
a. providing a sample obtained from the subject, wherein the sample comprises genomic DNA;
b. determining the nucleotide sequence of a fragment of the genomic DNA comprising at least a portion of the nucleotide sequence of the STMN2 gene set forth in SEQ ID NO: 1;
c. detecting in the nucleotide sequence of (b) the presence or absence of CA dinucleotides from position 30512 onwards in the nucleotide sequence of the STMN2 gene set forth in SEQ ID NO: 1;
d. identifying short (S) alleles and long (L) alleles in nucleotide sequences with CA dinucleotides in the STMN2 gene detected in (c), wherein short alleles have < 19 consecutive CA dinucleotides in the STMN2 gene, wherein long alleles have ≥ 19 consecutive CA dinucleotides in the STMN2 gene;
e. stratifying the subject for treatment of said ALS based on the presence of at least one short allele (S/L or S/S genotype) or the presence of two long alleles (L/L genotype);
f. treating the subject stratified in (e) with a treatment for said sporadic ALS.

30. The method of claim 29, wherein the nucleotide sequence of (b) is determined following amplification of the genomic DNA.

31. The method of claim 29 or claim 30, wherein the genomic DNA sample is amplified using oligonucleotide primers having the nucleotide sequences set forth in SEQ ID NO: 2 and 3, or functional fragments thereof.

32. The method of any one of claims 29 to 31, wherein the treatment for sporadic ALS is selected from the group consisting of riluzole, edaravone, medication for symptomatic relief, physical therapy, nutritional therapy and combinations thereof.

33. A kit for determining if a subject has, or has a genetic predisposition to developing, sporadic ALS, the kit comprising a probe or set of oligonucleotides designed to determine the nucleotide sequence of a fragment of genomic DNA comprising at least a portion of the nucleotide sequence of the STMN2 gene set forth in SEQ ID NO: 1.

34. The kit of claim 33, wherein the kit comprises oligonucleotide primers comprising the nucleotide sequences set forth in SEQ ID NO: 2 and 3, or functional fragments thereof.
